(19) United States
(12) Patent Application Publication
(10) Pub. No.: US 2026/0070961 A1
(43) Pub. Date: Mar. 12, 2026

(54) ANTI-DENV3 ANTIBODIES

(71) Applicant: Takeda Vaccines, Inc., Cambridge, MA (US)

(72) Inventors: David Dominguez, Cambridge, MA (US); Heather Watkins, Cambridge, MA (US); Isamu Tsuji, Yamaguchi (JP)

(73) Assignee: Takeda Vaccines, Inc., Cambridge, MA (US)

(21) Appl. No.: 19/102,737

(22) PCT Filed: Aug. 10, 2023
(86) PCT No.: PCT/US2023/072002

§ 371 Date: Feb. 10, 2025

Related U.S. Application Data
(60) Provisional application No. 63/371,263, filed on Aug. 12, 2022.

(51) Int. Cl. C07K 16/10; G01N 33/569

(52) U.S. Cl. CPC C07K 16/10; G01N 33/56983; C07K 2317/565; C07K 2317/76; C07K 2317/92; G01N 2333/185; G01N 2469/10; G01N 2470/10

(57) ABSTRACT

The present invention relates to anti-dengue virus serotype 3 (DENV3) antibodies and antigen binding fragments thereof. Further, nucleic acids encoding them and host cells comprising them are provided. In addition, the use of the antibodies in the prevention or treatment of dengue disease is provided. Also, diagnostic methods using them and kits comprising them are provided.

### CROSS REFERENCE TO RELATED APPLICATIONS

This application is an International Patent Cooperation Treaty (PCT) application claiming priority to, and the benefit of U.S. Provisional Application No. 63/371,263, filed Aug. 12, 2022, the entire contents of which are incorporated herein by reference in its entirety.

### SEQUENCE LISTING

This application incorporates by reference in its entirety the Sequence Listing entitled “T08596WO-Final_SQL.xml”, which was created on Aug. 2, 2023 and is 84 KB in size, and filed electronically herewith. The entire contents of the Sequence Listing are incorporated herein by reference.

### TECHNICAL FIELD OF THE INVENTION

The present invention relates to novel anti-dengue virus serotype 3 (DENV3) antibodies and antigen binding fragments thereof. Further, nucleic acids encoding them and host cells comprising them are provided. In addition, the use of the antibodies in the prevention or treatment of dengue disease is provided. Also, diagnostic methods using them and kits comprising them are provided.

### BACKGROUND OF THE INVENTION

Dengue disease is a mosquito-borne disease caused by infection with a dengue virus. Dengue virus infections can lead to debilitating and painful symptoms, including a sudden high fever, headaches, joint and muscle pain, nausea, vomiting and skin rashes. To date, four serotypes of dengue virus have been identified: dengue-1 (DENV-1), dengue-2 (DENV-2), dengue-3 (DENV-3) and dengue-4 (DENV-4). Dengue virus serotypes 1-4 can also cause dengue hemorrhagic fever (DHF) and dengue shock syndrome (DSS). In the most severe cases, DHF and DSS can be life threatening. Dengue viruses cause 50-100 million cases of debilitating dengue fever, 500,000 cases of DHF/DSS, and more than 20,000 deaths each year, a large portion of which are children. All four dengue virus serotypes are endemic throughout the tropical regions of the world and constitute the most significant mosquito-bome viral threat to humans there. Dengue viruses are transmitted to humans primarily by Aedes aegypti mosquitoes, but also by Aedes albopictus mosquitoes. Infection with one dengue virus serotype results in life-long protection from re-infection by that serotype, but does not prevent secondary infection by one of the other three dengue virus serotypes. In fact, previous infection with one dengue virus serotype may lead to an increased risk of severe disease (DHF/DSS) upon secondary infection with a different serotype.

To date, only one vaccine, Dengvaxia®, has been licensed for use in protecting against dengue disease. However, clinical trials have shown that Dengvaxia®; can enhance, rather than reduce, the risk of severe disease due to dengue infection in individuals who had not been previously infected by a dengue virus (seronegative populations). Therefore, Dengvaxia® is only recommended for use in individuals who had been previously infected with at least one dengue virus serotype (seropositive populations).

Anti-DENV3 antibodies are commercially available. However, these antibodies either do not exhibit a sufficient binding affinity and/or do not sufficiently neutralize the binding of the dengue virus to its target cell.

Therefore, there is a need for improved anti-DENV3 antibodies having increased binding affinity and/or neutralizing activity. There is a further need for antibodies being potentially suitable in the prevention or treatment of dengue disease.

### SUMMARY OF THE INVENTION

In a first aspect, the present invention provides an antibody specific for Dengue virus serotype 3 (DENV3) or an antigen binding fragment thereof, wherein
(i) the VH CDR1 region of the antibody or antigen binding fragment thereof is selected from the group consisting of SEQ ID NO: 5, SEQ ID NO: 10, SEQ ID NO: 15 and SEQ ID NO: 20, or a variant thereof having at least 85% identity;
(ii) the VH CDR2 region of the antibody or binding fragment thereof is selected from the group consisting of SEQ ID NO: 6, SEQ ID NO: 11, SEQ ID NO: 16 and SEQ ID NO: 21, or a variant thereof having at least 85% identity;
(iii) the VH CDR3 region of the antibody or antigen binding fragment thereof is selected from the group consisting of SEQ ID NO: 7, SEQ ID NO: 12, SEQ ID NO: 17 and SEQ ID NO 22, or a variant thereof having at least 85% identity;
(iv) the VL CDR1 region of the antibody or antigen binding fragment thereof is selected from the group consisting of SEQ ID NO: 8, SEQ ID NO: 13, SEQ ID NO: 18 and SEQ ID NO: 23, or a variant thereof having at least 82% identity;
(v) the VL CDR2 region of the antibody or antigen binding fragment thereof is selected from the group of an amino acid sequence consisting of RAS, LAS and GAS, or a variant thereof having at least 65% identity; and
(vi) the VL CDR3 region of the antibody or antigen binding fragment thereof is selected from the group consisting of SEQ ID NO: 9, SEQ ID NO: 14, SEQ ID NO: 19 and SEQ ID NO: 24, or a variant thereof having at least 85% identity, wherein the antibody or antigen binding fragment thereof has one or more of the following properties:
(1) a neutralization activity calculated as IC₅₀ value of 15 nM or less;
(2) a binding activity for DENV3-VLP calculated as EC₅₀ value of 60 ng/ml or less; and/or
(3) a k_{off} value of 1×10⁻⁴ sec⁻¹ or less.

In a second aspect the present invention provides nucleic acids encoding the antibody or antigen binding fragment thereof; vectors and host cells comprising them and methods for producing the antibodies or antigen binding fragments thereof.

In a third aspect, the present invention provides an in vitro method for detecting DENV3 viruses in a biological sample, wherein the method comprises contacting the antibody according to the present invention with a biological sample and determining the amount of antibody bound to the biological sample.

In a fourth aspect, the present invention provides a pharmaceutical formulation comprising the antibody according to the present invention.

In a fifth aspect, the present invention provides an antibody according to the present invention for use in the prevention or treatment of a Dengue disease in a subject.

In a sixth aspect, the present invention provides a method for the prevention or treatment of a Dengue disease in a subject comprising administering the antibody according to the present invention to the subject.

In a seventh aspect, the present invention provides a kit for the detection of DENV3 viruses comprising an antibody according to the present invention.

The present inventors have identified anti-DENV3 antibodies sharing common structural motifs, wherein the antibodies bind with high binding activity to intact DENV3 virus particles. In particular, the antibodies are characterized by (i) a strong neutralization activity as determined in a DENV3 reporter virus particle (RVP) assay, (ii) a strong binding activity against DENV3 as virus-like particle (VLP) and/or (iii) a low k_{off} value. In particular, the clones 8D4 and 12H6 had significantly improved neutralization activity. It was also surprisingly found that the clone 5D7 and 13E10 had significantly improved binding activity for DENV3-VLP.

The antibodies preferably show a serotype-specificity for DENV3 serotype with essentially no cross-reactivity for Zika virus.

A subgroup of the antibodies exhibits suitability for Western Blotting. It is unexpected and surprising that the present inventors could identify a structurally linked group of anti-DENV3 antibodies characterized by the above functional features.

### BRIEF DESCRIPTION OF THE DRAWINGS

FIGS. 1A-1P show the binding activity of the different antibodies to DENV3-VLP in a Luminex assay.

FIG. 2 shows the reactivity of the different antibodies to DENV3-VLP in a Western Blot analysis.

FIGS. 3A-3L show the neutralizing activity of the different antibodies in an RVP assay.

FIGS. 4A and 4B show the epitope binning of anti-DENV3 mAbs showing antibody bindings based on competitions.

FIGS. 5A-5C show the binding of different antibody clones to structure changed dengue virus.

### DETAILED DESCRIPTION OF THE INVENTION

In a first aspect the present invention provides an antibody specific for Dengue virus serotype 3 (DENV3) or an antigen binding fragment thereof, wherein
(i) the VH CDR1 region of the antibody or antigen binding fragment thereof is selected from the group consisting of SEQ ID NO: 5, SEQ ID NO: 10, SEQ ID NO: 15 and SEQ ID NO: 20, or a variant thereof having at least 85% identity;
(ii) the VH CDR2 region of the antibody or binding fragment thereof is selected from the group consisting of SEQ ID NO: 6, SEQ ID NO: 11, SEQ ID NO: 16 and SEQ ID NO: 21, or a variant thereof having at least 85% identity;
(iii) the VH CDR3 region of the antibody or antigen binding fragment thereof is selected from the group consisting of SEQ ID NO: 7, SEQ ID NO: 12, SEQ ID NO: 17 and SEQ ID NO 22, or a variant thereof having at least 85% identity;
(iv) the VL CDR1 region of the antibody or antigen binding fragment thereof is selected from the group consisting of SEQ ID NO: 8, SEQ ID NO: 13, SEQ ID NO: 18 and SEQ ID NO: 23, or a variant thereof having at least 82% identity;
(v) the VL CDR2 region of the antibody or antigen binding fragment thereof is selected from the group of an amino acid sequence consisting of RAS. LAS and GAS, or a variant thereof having at least 65% identity; and
(vi) the VL CDR3 region of the antibody or antigen binding fragment thereof is selected from the group consisting of SEQ ID NO: 9, SEQ ID NO: 14, SEQ ID NO: 19 and SEQ ID NO: 24, or a variant thereof having at least 85% identity, wherein the antibody or antigen binding fragment thereof has one or more of the following properties:
(1) a neutralization activity calculated as IC₅₀ value of 15 nM or less;
(2) a binding activity for DENV3-VLP calculated as EC₅₀ value of 60 ng/ml or less; and/or
(3) a k_{off} value of 1×10⁻⁴ sec⁻¹ or less.

“DENV3” includes any DENV serotype 3 virus strain. This serotype is segregated into subtypes I to IV. A detailed review can be found in Messer et al., Emerg. Infect. Diseases Vol. 9 (2003), pages 800-809. Preferably, the dengue virus strain is (16562 Philippines 1964).

“an antibody”: As is known in the art, an “antibody” is an immunoglobulin that binds specifically to a particular antigen. The term encompasses immunoglobulins that are naturally produced in that they are generated by an organism reacting to the antigen, and also those that are synthetically produced or engineered. An antibody may be monoclonal or polyclonal. An antibody may be a member of any immunoglobulin class, including any of the human classes: IgG, IgM, IgA, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CHI, CH2 and CH3). In some embodiments, the term “full length” is used in reference to an antibody to mean that it contains two heavy chains and two light chains, optionally associated by disulfide bonds as occurs with naturally-produced antibodies. In some embodiments, an antibody is produced by a cell. In some embodiments, an antibody is produced by chemical synthesis. In some embodiments, an antibody is derived from a mammal. In some embodiments, an antibody is derived from an animal such as, but not limited to, mouse, rat, horse, pig, or goat. In some embodiments, an antibody is produced using a recombinant cell culture system. In some embodiments, an antibody may be a purified antibody (for example, by immune-affinity chromatography). In some embodiments, an antibody may be a human antibody. In some embodiments, an antibody may be a humanized antibody (antibody from non-human species whose protein sequences have been modified to increase their similarity to antibody variants produced naturally in humans). In some embodiments, an antibody may be a chimeric antibody (antibody made by combining genetic material from anon-human source, e.g., mouse, rat, horse, or pig, with genetic material from humans).

“specific for DENV3” herein means that the antibody or antigen binding fragment significantly binds to an antigen of dengue virus serotype 3 as compared to a non-specific background. The skilled person is aware of several techniques for testing specific binding of an antibody. The antigen to which the antibody binds may be a structural or non-structural protein of dengue virus. Preferably, the antigen is the envelope protein of dengue virus. The envelope protein is characterized by three structural domains, EI, EII and EIII. More preferably, the antibody or antigen binding fragment thereof binds specifically to domain III of the dengue envelope protein.

“an antigen binding fragment thereof”: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids. Preferably, the antigen binding fragment thereof can be obtained by screening of fragments for specific binding to the dengue envelope protein, more preferably for the domain III of the dengue envelope protein.

The variant of the indicated CDR sequences has an identity of at least 85%, preferably of at least 90% and even more preferred of at least 95%, and most preferred of at least 98% compared to the indicated CDR sequences. A variant includes one or more amino acid substitution, insertion and/or deletion compared to the indicated CDR sequences.

“neutralization activity” herein means the capacity to neutralize the binding of the dengue virus, in particular DENV3, to a target cell. Preferably, the target cell is a human cell. The neutralization activity may be determined in vitro or in vivo. Suitable neutralization assays are known to the skilled person. The neutralization assay may be a microneutralization (MNT) assay or an reporter virus particle (RVP) assay. Suitable MNT assays are described in WO 2020/051328. A suitable RVP assay is described in the examples. Preferably, the target cell for the RVP assay is Raji DC-SIGN cell (Accellerate, Hamburg, Germany). The used reporter virus particle is a DENV3 RVP. Such a RVP is commercially available e.g. from Integral Molecular. Preferably, the DENV3 RVP is derived from the DENV3 strain 16562/Philippines/1964. The “IC₅₀ value” refers to the antibody concentration at which 50% of the infection of the target cells is observed. The lower the IC₅₀ value, the higher is the neutralization activity of the antibody.

In a preferred embodiment the antibodies according to the present invention have an IC₅₀ value of 15 nM or less, preferably the IC₅₀ value is less than 5 nM or less, more preferred, 2 nM or less and even more preferred of 1 nM or less, in particular 0.5 nM or less.

“binding activity for DENV3-VLP”: DENV3-VLP has been selected as a model for intact dengue virus serotype 3 particles; see also Metz et al., Virol. J. 15 (2018), 60. It is considered that the three-dimensional form of the proteins on the surface of the VLP is in the native form correspond to the three-dimensional form of the proteins on the surface of the live virus. The skilled person knows how to produce dengue virus VLPs. DENV3 VLPs are also commercially available from e.g. the company Native Antigen. Preferably, the DENV3 VLP is derived from the DENV3 strain Sri Lanka D3/H/IMTSSA-SRI/2000/1266. The VLP may be attached to a surface of a plate for carrying out the assay. Suitable assay forms include RIA, ELISA, or a chemiluminescent assay. Preferably, the binding of the antibody or antigen binding fragment thereof is determined in a Luminex assay format (Nascimento et al. Development and Characterization of a Multiplex Assay to Quantify Complement-Fixing Antibodies against Dengue Virus. Int J Mol Sci 22 (2021), 12004). One suitable format is described in the examples. The binding activity is calculated as EC₅₀ value. The EC₅₀ value corresponds to the antibody concentration, wherein 50% of the maximal binding of the antibody is observed.

In a further preferred embodiment the antibodies according to the present invention have a binding activity calculated as EC₅₀ value of 50 ng/ml or less, and even more preferred of 30 ng/ml or less.

Binding kinetics relates to the rate at which the binding sites at a molecule such as an antibody are occupied with the ligand molecules such as antigens, i.e. the formation of the binding complex (association rate kₒₙ) and to the rate at which the ligand molecules are released from the binding sites, i.e. the dissociation of the binding complex (dissociation rate k_{off}). In the following k_{off} is also termed k_{off} value.

According to a preferred embodiment the association rate kₒₙ is measured in a biosensor format such as surface plasmon resonance (SPR) or biolayer interferometry (BLI). Preferably, BLI is used. The association rate is measured when the binding sites attached to the biosensor are contacted with a solution containing the ligand molecules. According to a preferred embodiment the dissociation rate k_{off} is measured when the biosensor with the binding complex is removed from the above solution and introduced into a solution which does not contain the ligand molecules such as a buffer solution. Methods for measuring the k_{off} value are described e.g. in WO 2021/067714 A2. Preferably, the k_{off} value is determined as described in the present examples.

Preferably, the kₒₙ value of an antibody or antigen binding fragment thereof is determined using DENV3-VLP as binding partner.

In a further preferred embodiment the antibodies according to the present invention exhibit a k_{off} value of 1×10 sec⁻¹ or less, preferably of 5×10⁻⁵ sec⁻¹ or less, and more preferred of 2×10⁻⁵ sec⁻¹ or less.
“the antibody is not cross-reactive with Zika virus” herein means that in a preferred embodiment the antibody or antibody fragment thereof according to the invention does essentially not bind to Zika virus. The skilled person is aware of methods for testing the binding of antibodies to antigens. Suitable assays include, but are not limited to ELISA, RIA, luminex assay and avidity assay. As antigen, the Zika virus may be bound to the plate surface. Alternatively, Zika virus VLP (virus-like particle) may be used. The prior art anti-DENV3 antibodies are characterized by cross-reactivity to Zika virus which impairs for example diagnostic applications to distinguish superinfections by a dengue virus and a Zika virus from a single dengue infection. This drawback of the prior art is overcome by this preferred embodiment. A further application may be as control in the development of dengue specific vaccines.
“competitive assay” In competitive immunoassays the analyte and the labeled analyte (tracer) are mixed with a limited amount of anti-analyte antibody. After incubation for a certain period, the bound or the free fraction of the tracer is measured and related to the concentration of the analyte in the sample. Suitable assays include, but are not limited to ELISA, RIA, luminex assay and avidity assay.

In a further preferred embodiment the amino acid sequence of the VH chain of the antibody or antigen binding fragment has an identity of at least 80% compared to the amino acid sequence set forth in SEQ ID NO: 3, preferably at least 90%; and even more preferred at least 95% sequence identity.

In a further preferred embodiment the amino acid sequence of the VL chain of the antibody or antigen binding fragment has an identity of at least 80% compared to the amino acid sequence set forth in SEQ ID NO: 4, preferably at least 90%; and even more preferred at least 95% sequence identity.

In a further preferred embodiment the antibody of antigen binding fragment thereof does not cross-react with any dengue serotype other than DENV3, preferably, the antibody or antigen binding fragment does not cross-react with Zikavirus.

Particularly preferred are the following combinations of VH CDR1 to CDR3 and VL CDR1 to CDR3. These combinations are based on the antibody clones identified in the examples as 8D4, 12H6, 5D7 and 13E10, respectively.

In a further preferred embodiment the antibody or antigen binding fragment thereof comprises the heavy chain CDR regions 1 to 3 having the amino acid sequences of SEQ ID Nos: 5 to 7, respectively, and the light chain CDR region 1 has the amino acid sequence of SEQ ID NO: 8. The light chain CDR region 2 has the amino acid sequence RAS, and the light chain CDR region 3 has the amino acid sequence SEQ ID NO: 9. Preferably, the above CDR regions are in combination with the FWR regions identified in the below Tables l a and 2a for the particular clones 8D4, 12H6, 5D7 and 13E10, respectively.

In a further preferred embodiment the antibody or antigen binding fragment thereof comprises the heavy chain CDR regions 1 to 3 having the amino acid sequences of SEQ ID Nos: 10 to 12, respectively, and the light chain CDR region 1 has the amino acid sequence of SEQ ID NO: 13. The light chain CDR region 2 has the amino acid sequence LAS, and the light chain CDR region 3 has the amino acid sequence SEQ ID NO: 14.

In a further preferred embodiment the antibody or antigen binding fragment thereof comprises the heavy chain CDR regions 1 to 3 having the amino acid sequences of SEQ ID Nos: 15 to 17, respectively, and the light chain CDR region 1 has the amino acid sequence of SEQ ID NO: 18. The light chain CDR region 2 has the amino acid sequence RAS, and the light chain CDR region 3 has the amino acid sequence SEQ ID NO: 19.

In a further preferred embodiment the antibody or antigen binding fragment thereof comprises the heavy chain CDR regions 1 to 3 having the aKino acid sequences of SEQ ID Nos: 20 to 22, respectively, and the light chain CDR region 1 has the amino acid sequence of SEQ ID NO: 23. The light chain CDR region 2 has the amino acid sequence GAS, and the light chain CDR region 3 has the amino acid sequence SEQ ID NO: 24.

TABLE 1
VH CDR1 to CDR3 Sequences

| Clone | CDR1 | CDR2 | CDR3 |
|---|---|---|---|
| DENV3-5D7 | GFSFSSGYD (SEQ | IYITDTGSS | AKTNYGSGGFAFNL (SEQ |
| | ID NO: 15) | (SEQ ID NO: | ID NO: 17) |
| | | 16) | |
| DENV3-8D4 | GFSFSNVYY (SEQ | IGTGDGNT | ARDIYSYDNTGVYTVPKL |
| | ID No: 5) | (SEQ ID NO: | (SEQ ID NO: 7) |
| | | 6) | |
| DENV3-12H6 | GIDFSSSYW (SEQ | IYTGSSTT | ARFYDVGVYFNL (SEQ ID |
| | ID NO: 10) | (SEQ ID NO: | NO: 12) |
| | | 11) | |
| DENV3- | GFSFSSGSY (SEQ ID | IYSGGDVT | ARGVGTYNYAGYAYPYYFSL |
| 13E10 | NO: 20) | (SEQ ID NO: | (SEQ ID NO: 22) |
| | | 21) | |

TABLE 1a
VH FWR1 to FWR4 Sequences

| clone | FWR1 | FWR2 | FWR3 | FWR4 |
|---|---|---|---|---|
| DENV3-5D7 | QSLEESGGDLVK | MCWVRQAP | YYATWARGRFTISKTSSTS | WGPGTLVTVSS (SEQ ID |
| | PGASLTLTCTAS | GKGLEWIAC | VTLQVTSLTAADTATYFC | NO: 64) |
| | (SEQ ID NO: 61) | (SEQ ID NO: | (SEQ ID NO: 63) | |
| | | 62) | | |
| DENV3-8D4 | QEQLEESGGGLV | ICWVRQAPG | WYASWAKGRFTISKASST | WGPGTLVTVSS (SEQ ID |
| | KPGGTLTLTCTAS | KGLEWIGC | TVILQVTSLTAADTATYFC | NO: 72) |
| | (SEQ ID NO: 69) | (SEQ ID NO: | (SEQ ID NO: 71) | |
| | | 70) | | |
| DENV3-12H6 | QEQLEESGGDLV | ICWVRQAPG | WYAAWAKGRFTISKPSST | WGPGTLVTVSS (SEQ ID |
| | KPEGSLTLTCKAS | KGLEWIGC | TVTLQMTSLTAADTATYFC | NO: 80) |
| | (SEQ ID NO: 77) | (SEQ ID NO: | (SEQ ID NO: 79) | |
| | | 78) | | |
| DENV3- | QSLEESGGDLVK | MCWVRQAP | YYTSWAKGRFTISRTSSTT | WGPGTLVTVSS (SEQ ID |
| 13E10 | PGASLTLTCKAS | GKGLDWIAC | VTLQMTSLTAADTATYFC | NO: 88) |
| | (SEQ ID NO: 85) | (SEQ ID NO: | (SEQ ID NO: 87) | |
| | | 86) | | |

TABLE 2
VL CDR1 to CDR3 sequences

| clone | CDR1 | CDR2 | CDR3 |
|---|---|---|---|
| DENV3-5D7 | QSISNL | RAS | QCTYGGSSITNWA |
| | (SEQ ID | | (SEQ ID NO: 19) |
| | NO: 18) | | |
| DENV3-8D4 | ESVYSNNR | RAS | AGGYSGSSDKG |
| | (SEQ ID | | (SEQ ID NO: 9) |
| | NO: 8) | | |
| DENV3-12H6 | ESISSW | LAS | AGYKSSVTDGFA |
| | (SEQ ID | | (SEQ ID NO: 14) |
| | NO: 13) | | |
| DENV3- | ENVYGS | GAS | QGAYYASNFDAT |
| 13E10 | (SEQ ID | | (SEQ ID NO: 24) |
| | NO: 23) | | |

TABLE 2a
VL FWR1 to FWR4 sequences

| clone | FWR1 | FWR2 | FWR3 | FWR4 |
|---|---|---|---|---|
| DENV3-5D7 | DVVMTQTPASVEARVG | LAWYQQKPGQ | ALESGVPSRFRG | FGGGTEVVVK |
| | GTVTINCQAS (SEQ ID | PPKLLIY (SEQ ID | SGSGTEFTLTISD | (SEQ ID NO: |
| | NO: 65) | NO: 66) | LECADAATYYC | 68) |
| | | | (SEQ ID NO: 67) | |
| DENV3-8D4 | AAVLTQTPSPVSAAVGG | LAWYQQKPGQ | TLESGVPSRFKGS | FGGGTEVVVK |
| | TVSISCQSS (SEQ ID NO: | PPKLLIY (SEQ ID | GSGTEFTLTISDV | (SEQ ID NO: |
| | 73) | NO: 74) | RCDDAATYYC | 76) |
| | | | (SEQ ID NO: 75) | |
| DENV3-12H6 | AIVMTQTPSSKSVPVGD | LAWYQQKPGQ | TLASGVPSRFKGS | FGGGTEVVVK |
| | TVTINCQAS (SEQ ID | PPKLLIY (SEQ ID | GSGTQFTLTISDV | (SEQ ID NO: |
| | NO: 81) | NO: 82) | VCDDAATYYC | 84) |
| | | | (SEQ ID NO: 83) | |
| DENV3-13E10 | ALVMTQTPSSVSEPVGG | LAWYQQKPGQ | YLASGVPSRFGG | FGGGTEVVVK |
| | TVAINCQAS (SEQ ID | PPKLLIY (SEQ ID | SGSGTEFTLTISD | (SEQ ID NO: |
| | NO: 89) | NO: 90) | LECADAATYYC | 92) |
| | | | (SEQ ID NO: 91) | |

TABLE 3
VH amino acid sequences of particular clones

| Clone | |
|---|---|
| 8D4 | QEQLEESGGGLVKPGGTLTLTCTASGFSFSNVYYICWVRQAP |
| | GKGLEWIGCIGTGDGNTWYASWAKGRFTISKASSTTVILQVT |
| | SLTAADTATYFCARDIYSYDNTGVYTVPKLWGPGTLVTVSS |
| | (SEQ ID NO: 25) |
| 12H6 | QEQLEESGGDLVKPEGSLTLTCKASGIDFSSSYWICWVRQAP |
| | GKGLEWIGCIYTGSSTTWYAAWAKGRFTISKPSSTTVTLQMT |
| | SLTAADTATYFCARFYDVGVYFNLWGPGTLVTVSS (SEQ |
| | ID NO: 27) |
| 5D7 | QSLEESGGDLVKPGASLTLTCTASGFSFSSGYDMCWVRQAPG |
| | KGLEWIACIYITDTGSSYYATWARGRFTISKTSSTSVTLQVT |
| | SLTAADTATYFCAKTNYGSGGFAFNLWGPGTLVTVSS (SEQ |
| | ID NO: 29) |
| 13E10 | QSLEESGGDLVKPGASLTLTCKASGFSFSSGSYMCWVRQAPG |
| | KGLDWIACIYSGGDVTYYTSWAKGRFTISRTSSTTVTLQMTS |
| | LTAADTATYFCARGVGTYNYAGYAYPYYFSLWGPGTLVTVSS |
| | (SEQ ID NO: 31) |

TABLE 4
VL amino acid sequences of particular clones

| Clone | |
|---|---|
| 8D4 | AAVLTQTPSPVSAAVGGTVSISCQSSESVYSNNRLAWYQQKP |
| | GQPPKLLIYRASTLESGVPSRFKGSGSGTEFTLTISDVRCDD |
| | AATYYCAGGYSGSSDKGFGGGTEVVVK (SEQ ID NO: 26) |
| 12H6 | AIVMTQTPSSKSVPVGDTVTINCQASESISSWLAWYQQKPGQ |
| | PPKLLIYLASTLASGVPSRFKGSGSGTQFTLTISDVVCDDAA |
| | TYYCAGYKSSVTDGFAFGGGTEVVVK (SEQ ID NO: 28) |
| 5D7 | DVVMTQTPASVEARVGGTVTINCQASQSISNLLAWYQQKPGQ |
| | PPKLLIYRASALESGVPSRFRGSGSGTEFTLTISDLECADAA |
| | TYYCQCTYGGSSITNWAFGGGTEVVVK (SEQ ID NO: 30) |
| 13E10 | ALVMTQTPSSVSEPVGGTVAINCQASENVYGSLAWYQQKPGQ |
| | PPKLLIYGASYLASGVPSRFGGSGSGTEFTLTISDLECADAA |
| | TYYCQGAYYASNFDATFGGGTEVVVK (SEQ ID NO: 32) |

TABLE 5
nucleic acid sequences encoding VH CDR1 to CDR3

| clone | CDR1 | CDR2 | CDR3 |
|---|---|---|---|
| DENV3-8D4 | GGATTCTCCTTCAGTAACGT | ATTGGTACTGGTGATGGCA | GCGAGAGATATATATAGT |
| | CTACTAC (SEQ ID NO: 33) | ACACA (SEQ ID NO: 34) | TATGATAATACTGGTGTTT |
| | | | ATACTGTTCCTAAGTTG |
| | | | (SEQ ID NO: 35) |
| DENV3-12H6 | GGAATCGACTTCAGTAGCA | ATTTATACTGGTAGTAGTAC | GCGAGATTTTATGATGTCG |
| | GCTACTGG (SEQ ID NO: 38) | TACA (SEQ ID NO: 39) | GTGTTTACTTTAACTTG |
| | | | (SEQ ID NO: 40) |
| DENV3-5D7 | GGATTCTCCTTCAGTAGCGG | ATTTATATTACTGATACTGG | GCGAAAACTAATTATGGT |
| | CTACGAC (SEQ ID NO: 43) | TAGCTCT (SEQ ID NO: 44) | AGTGGTGGTTTTGCCTTTA |
| | | | ACTTG (SEQ ID NO: 45) |
| DENV3-13E10 | GGCTTCTCCTTCAGTAGCGG | ATTTATAGTGGTGGTGATGT | GCGAGAGGGGTCGGTACT |
| | CTCATAC (SEQ ID NO: 48) | CACT (SEQ ID NO: 49) | TATAATTATGCTGGTTATG |
| | | | CTTATCCATACTACTTTAG |
| | | | CTTG (SEQ ID NO: 50) |

TABLE 6
nucleic acid sequences encoding VL CDR1 to CDR3

| clone | CDR1 | CDR2 | CDR3 |
|---|---|---|---|
| DENV3- | GAGAGTGTTTATAGTAAC | AGGGCATC | GCAGGCGGCTATAGTGGTAGTAGTGATAAAGG |
| 8D4 | AACCGC (SEQ ID NO: 36) | C | T (SEQ ID NO: 37) |
| DENV3- | GAGAGCATTAGCAGTTGG | CTGGCATC | GCAGGATATAAAAGTAGTGTTACTGATGGTTTT |
| 12H6 | (SEQ ID NO: 41) | C | GCT (SEQ ID NO: 42) |
| DENV3- | CAGAGCATTAGCAACCTC | AGGGCTTC | CAATGCACTTATGGTGGTAGTAGTATTACTAAT |
| 5D7 | (SEQ ID NO: 46) | C | TGGGCT (SEQ ID NO: 47) |
| DENV3- | GAGAACGTTTACGGCTCT | GGTGCATC | CAAGGCGCTTATTATGCCAGTAATTTTGATGCT |
| 13E10 | (SEQ ID NO: 51) | C | ACT (SEQ ID NO: 52) |

TABLE 7
Nucleic acid sequences encoding VH chain of particular clones

| Clone | |
|---|---|
| 8D4 | CAGGAGCAGCTGGAGGAGTCCGGGGGAGGCCTGGTCAAGCCTGGA |
| | GGAACCCTGACACTCACCTGCACAGCTTCTGGATTCTCCTTCAGTA |
| | ACGTCTACTACATCTGCTGGGTCCGCCAGGCTCCAGGGAAGGGGCT |
| | GGAATGGATCGGATGCATTGGTACTGGTGATGGCAACACATGGTA |
| | CGCGAGCTGGGCGAAAGGCCGATTCACCATCTCCAAAGCCTCGTC |
| | GACCACGGTGATTCTGCAAGTGACCAGTCTGACAGCCGCGGACAC |
| | GGCCACCTATTTCTGTGCGAGAGATATATATAGTTATGATAATACT |
| | GGTGTTTATACTGTTCCTAAGTIGTGGGGCCCAGGCACCCTGGTCA |
| | CCGTCTCCTCAG (SEQ ID NO: 53) |
| 12H6 | CAGGAGCAGCTGGAGGAGTCCGGGGGAGACCTGGTCAAGCCTGAG |
| | GGATCCCTGACACTCACCTGCAAAGCCTCTGGAATCGACTTCAGTA |
| | GCAGCTACTGGATATGCTGGGTCCGCCAGGCTCCAGGGAAGGGGC |
| | TGGAGTGGATCGGATGCATTTATACTGGTAGTAGTACTACATGGTA |
| | CGCGGCCTGGGCGAAAGGCCGATTCACCATCTCCAAGCCCTCGTCG |
| | ACCACGGTGACTCTGCAAATGACCAGTCTGACGGCCGCGGACACG |
| | GCCACGTATTTCTGTGCGAGATTTTATGATGTCGGTGTTTACTTTAA |
| | CTTGTGGGGCCCAGGCACCCTGGTCACCGTCTCCTCAG (SEQ ID NO: |
| | 55) |
| 5D7 | CAGTCGTTGGAGGAGTCCGGGGGAGACCTGGTCAAGCCTGGGGCA |
| | TCCCTGACACTCACCTGCACAGCCTCTGGATTCTCCTTCAGTAGCG |
| | GCTACGACATGTGCTGGGTCCGCCAGGCTCCAGGGAAGGGGTTGG |
| | AGTGGATCGCATGCATTTATATTACTGATACTGGTAGCTCTTATTAC |
| | GCGACCTGGGCGAGAGGCCGATTCACCATCTCCAAAACCTCGTCG |
| | ACCTCGGTGACTCTGCAAGTGACCAGTCTGACAGCCGCGGACACG |
| | GCCACCTATTTCTGTGCGAAAACTAATTATGGTAGTGGTGGTTTTG |
| | CCTTTAACTTGTGGGGCCCAGGCACCCTGGTCACCGTCTCCTCAG |
| | (SEQ ID NO: 57) |
| 13E10 | CAGTCGTTGGAGGAGTCCGGGGGAGACCTGGTCAAGCCTGGGGCA |
| | TCCCTGACACTCACCTGCAAAGCCTCTGGCTTCTCCTTCAGTAGCG |
| | GCTCATACATGTGCTGGGTCCGCCAGGCTCCAGGGAAGGGGCTGG |
| | ATTGGATCGCATGCATTTATAGTGGTGGTGATGTCACTTACTACAC |
| | GAGCTGGGCGAAAGGCCGATTCACCATCTCCAGAACCTCGTCGAC |
| | CACGGTGACTCTGCAAATGACCAGTCTGACAGCCGCGGACACGGC |
| | CACCTATTTCTGTGCGAGAGGGGTCGGTACTTATAATTATGCTGGT |
| | TATGCTTATCCATACTACTTTAGCTTGTGGGGCCCAGGCACCCTGG |
| | TCACCGTCTCCTCAG (SEQ ID NO: 59) |

TABLE 8
Nucleic acid sequences encoding VL chain of particular clones

| Clone | |
|---|---|
| 8D4 | GCCGCCGTGCTGACCCAGACTCCATCTCCCGTGTCTGCAGCTGTGG |
| | GAGGCACAGTCAGCATCAGTTGCCAGTCCAGTGAGAGTGTTTATAG |
| | TAACAACCGCTTAGCCTGGTATCAGCAGAAACCAGGGCAGCCTCC |
| | CAAACTCCTGATCTACAGGGCATCCACTCTGGAATCTGGGGTCCCA |
| | TCGCGGTTCAAAGGCAGTGGATCTGGGACAGAGTTCACTCTCACCA |
| | TCAGCGACGTGCGGTGTGACGATGCTGCCACTTATTACTGTGCAGG |
| | CGGCTATAGTGGTAGTAGTGATAAAGGTTTCGGCGGAGGGACCGA |
| | GGTGGTGGTCAAAG (SEQ ID NO: 54) |
| 12H6 | GCCATCGTGATGACCCAGACTCCATCTTCCAAGTCTGTCCCTGTGG |
| | GAGACACAGTCACCATCAATTGCCAGGCCAGTGAGAGCATTAGCA |
| | GTTGGTTAGCCTGGTATCAGCAGAAACCAGGGCAGCCTCCCAAGCT |
| | CCTGATCTATCTGGCATCCACTCTGGCATCTGGGGTCCCATCGCGG |
| | TTCAAAGGCAGTGGATCTGGGACACAGTTCACTCTCACCATCAGCG |
| | ATGTGGTGTGTGACGATGCTGCCACTTACTACTGTGCAGGATATAA |
| | AAGTAGTGTTACTGATGGTTTTGCTTTCGGCGGAGGGACCGAGGTG |
| | GTGGTCAAAG (SEQ ID NO: 56) |
| 5D7 | GATGTTGTGATGACCCAGACTCCAGCCTCCGTGGAGGCACGTGTGG |
| | GAGGCACAGTCACCATCAATTGCCAGGCCAGTCAGAGCATTAGCA |
| | ACCTCTTAGCCTGGTATCAGCAGAAACCAGGGCAGCCTCCCAAGCT |
| | CCTGATCTACAGGGCTTCCGCTCTGGAATCTGGGGTCCCGTCGCGG |
| | TTCAGAGGCAGTGGATCTGGGACAGAGTTCACTCTCACCATCAGCG |
| | ACCTGGAGTGTGCCGATGCTGCCACTTACTACTGTCAATGCACTTA |
| | TGGTGGTAGTAGTATTACTAATTGGGCTTTCGGCGGAGGGACCGAG |
| | GTGGTGGTCAAAG (SEQ ID NO: 58) |
| 13E10 | GCCCTTGTGATGACCCAGACTCCATCCTCCGTGTCTGAACCTGTGG |
| | GAGGCACAGTCGCCATCAATTGCCAGGCCAGTGAGAACGTTTACG |
| | GCTCTTTAGCCTGGTATCAGCAGAAACCAGGGCAGCCTCCCAAGCT |
| | CCTGATCTATGGTGCATCCTATCTGGCATCTGGGGTCCCATCGCGG |
| | TTCGGTGGCAGTGGATCTGGGACAGAGTTCACTCTCACCATCAGCG |
| | ACCTGGAGTGTGCCGATGCAGCCACTTACTACTGTCAAGGCGCTTA |
| | TTATGCCAGTAATTTTGATGCTACTTTCGGCGGAGGGACCGAGGTG |
| | GTGGTCAAAG (SEQ ID NO: 60) |

In a more preferred embodiment the antibody or antigen binding fragment thereof comprises in the VH domain the amino acid sequence set forth in SEQ ID NO: 25 or a derivative thereof, and in the VL domain the amino acid sequence set forth in SEQ ID NO: 26 or a derivative thereof, wherein the derivative differs from the indicated sequence by not more than five amino acid mutations. Even more preferred, the derivative differs by not more than three amino acid mutations from the indicated sequence, in particular not more than one amino acid mutation from the indicated sequence.

In a more preferred embodiment the antibody or antigen binding fragment thereof comprises in the VH domain the amino acid sequence set forth in SEQ ID NO: 27 or a derivative thereof, and in the VL domain the amino acid sequence set forth in SEQ ID NO: 28 or a derivative thereof, wherein the derivative differs from the indicated sequence by not more than five amino acid mutations. Even more preferred, the derivative differs by not more than three amino acid mutations from the indicated sequence, in particular not more than one amino acid mutation from the indicated sequence.

In a more preferred embodiment the antibody or antigen binding fragment thereof comprises in the VH domain the amino acid sequence set forth in SEQ ID NO: 29 or a derivative thereof, and in the VL domain the amino acid sequence set forth in SEQ ID NO: 30 or a derivative thereof, wherein the derivative differs from the indicated sequence by not more than five amino acid mutations. Even more preferred, the derivative differs by not more than three amino acid mutations from the indicated sequence, in particular not more than one amino acid mutation from the indicated sequence.

In a more preferred embodiment the antibody or antigen binding fragment thereof comprises in the VH domain the amino acid sequence set forth in SEQ ID NO: 31 or a derivative thereof, and in the VL domain the amino acid sequence set forth in SEQ ID NO: 32 or a derivative thereof, wherein the derivative differs from the indicated sequence by not more than five amino acid mutations. Even more preferred, the derivative differs by not more than three amino acid mutations from the indicated sequence, in particular not more than one amino acid mutation from the indicated sequence.

The amino acid mutations may be selected from one or more additions, deletions and/or substitutions.

In a further aspect a pair of nucleic acids comprising a first nucleic acid encoding the heavy chain of the antibody or antigen binding fragment and a second nucleic acid encoding the light chain of the antibody or antigen binding fragment is provided. Further, a vector comprising the first nucleic acid of and/or the second nucleic acid under the control of one or more suitable promoters is provided. The promoter may be a constitutive or inducible promoter. Suitable promoters are known to the skilled person.

In addition, a host cell transformed with at least one vector of the present invention and capable of expressing the antibody or antigen binding fragment is provided.

In a further aspect a method for the recombinant production of the antibody comprising culturing the transformed host cell under conditions suitable of expressing the antibody and optionally purifying the antibody from the culture medium is provided.

Methods for generating antibodies (e.g., monoclonal antibodies and/or polyclonal antibodies) are well known in the art. It will be appreciated that a wide range of animal species can be used for the production of antisera, including rabbit, mouse, rat, hamster, guinea pig or goat. The choice of animal may be decided upon the ease of manipulation, costs or the desired amount of sera, as would be known to one of skill in the art. It will be appreciated that the antibody or antigen binding fragment thereof can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

The antibody or antigen binding fragment thereof may be produced, for example, by utilizing a host cell system engineered to express an inventive antibody-encoding nucleic acid. Alternatively or additionally, provided antibody agents may be partially or fully prepared by chemical synthesis (e.g., using an automated peptide synthesizer).

Exemplary sources for the antibody or antigen binding fragment thereof suitable for the invention include, but are not limited to, conditioned culture medium derived from culturing a recombinant cell line that expresses a protein of interest, or from a cell extract of, e.g., antibody-producing cells, bacteria, fungal cells, insect cells, transgenic plants or plant cells, transgenic animals or animal cells, or serum of animals, ascites fluid, hybridoma or myeloma supernatants. Suitable bacterial cells include, but are not limited to, Escherichia coli cells. Examples of suitable E. coli strains include: HB101, DH5a, GM2929, JM109, KW251, NM538, NM539, and any E. coli strain that fails to cleave foreign DNA. Suitable fungal host cells that can be used include, but are not limited to, Saccharomyces cerevisiae, Pichia pastoris and Aspergillus cells. Suitable insect cells include, but are not limited to, S2 Schneider cells, D. Mel-2 cells, SF9, SF21, High-5™, Mimic™-SF9, MG1 and KCl cells. Suitable exemplary recombinant cell lines include, but are not limited to, BALB/c mouse myeloma line, human retinoblasts (PER.C6), monkey kidney cells, human embryonic kidney line (293), baby hamster kidney cells (BHK), Chinese hamster ovary cells (CHO), mouse Sertoli cells, African green monkey kidney cells (VERO-76), human cervical carcinoma cells (HeLa), canine kidney cells, buffalo rat liver cells, human lung cells, human liver cells, mouse mammary tumor cells, TRI cells, MRC 5 cells, FS4 cells, and human hepatoma line (Hep G2).

The antibody or antigen binding fragment thereof can be expressed using various vectors (e.g., viral vectors) known in the art and cells can be cultured under various conditions known in the art (e.g., fed-batch). Various methods of genetically engineering cells to produce antibodies are well known in the art. See e.g. Ausabel et al, eds. (1990), Current Protocols in Molecular Biology (Wiley, New York).

The antibody or antigen binding fragment thereof may be purified, if desired, using filtration, centrifugation and/or various chromatographic methods such as HPLC or affinity chromatography. In some embodiments, fragments of the provided antibodies are obtained by methods which include digestion with enzymes, such as pepsin or papain, and/or by cleavage of disulfide bonds by chemical reduction.

The antibody or antigen binding fragment thereof may be coupled to a detectable marker.

In a further aspect an in vitro method for detecting DENV3 viruses in a biological sample is provided, wherein the method comprises contacting the antibody with a biological sample and determining the amount of antibody bound to the biological sample. The in vitro method may be an enzymatic, fluorescent or chemiluminescent method. In a preferred embodiment, if the antibody or antibody fragment is not cross-reactive with Zika virus, it may be used as a diagnostic assay to distinguish a dengue monoinfection from a superinfection with dengue virus and Zika virus.

In a further aspect a kit for the detection of DENV3 viruses comprising an antibody or antigen binding fragment according to the present invention is provided. The kit may also contain instructions for carrying out the detection process.

In a further aspect a pharmaceutical formulation comprising the antibody or an antigen binding fragment thereof and optionally one or more pharmaceutically acceptable carrier is provided. As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

In a further aspect an antibody or antigen binding fragment according to the present invention is provided for use in the prevention or treatment of a Dengue disease in a subject. The antibody or antigen binding fragment may be present in a therapeutically effective amount. As used herein, the term “therapeutically effective amount” refers to an amount of a therapeutic protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

In a further aspect a method for the prevention or treatment of a Dengue disease in a subject is provided, wherein the method comprises administering the antibody or antigen binding fragment of the present invention. The above considerations for the use of the antibody or antigen binding fragment for the prevention or treatment of dengue disease apply equally. The subject is preferably a human.

### Examples

### Materials and Methods

### Antigens and Other Reagents

All VLP and proteins were purchased from The Native Antigen Company (Oxford, U.K.: Table 9).

TABLE 9
VLP and inactivated virus for immunized antigens

| | | | Accession |
|---|---|---|---|
| Description | Source | Strain info | No |
| DENV-2 VLP | Native | Thailand/16681/84 | AAP06254.1 |
| | Antigen | | |
| DENV-3 VLP | Native | Sri Lanka D3/H/IMTSSA- | AAM51537.1 |
| | Antigen | SRI/2000/1266 | |
| DENV-4 VLP | Native | Dominica/814669/1981 | P09866.2 |
| | Antigen | | |
| DENV2 | Microbix | Thailand/16681/84 | AAB58782.1 |
| inactivated | | | |
| virus | | | |

Inactivated dengue viruses were obtained from Microbix Biosystems (Mississauga, ON, Canada). RVP was purchased from Integral Molecular (Philadelphia PE, USA: Table 10).

TABLE 10
RVP reagents

| Description | Source | Strain info | Accession No |
|---|---|---|---|
| DENV-1 RVP | Intrgral | 16007/Thailand/1964 | QTX92473.1 |
| | Molecular | | |
| DENV-2 RVP | Intrgral | 16681/Thailand/1964 | NP_056776.2 |
| | Molecular | | |
| DENV-3 RVP | Intrgral | 16562/Philippines/1964 | U11673.1 |
| | Molecular | | |

Assay-Ready Raji DC-SIGN Cells (ARC) were purchased from Accellerate (Hamburg, Germany) [1] 5J7 and Mab513 was obtained from Creative Biolabs (Shirley, NY), DV10, DV18, DV63, DV78 and 4G2 were purchased from Absolute antibody (Oxford, UK) 777-3 (D6-8A1-12), 78-2 was expressed and purified from CHO cell, hybridoma cells and Expi293 cells. respectively (Table 11).

TABLE 11
Commercial and purified mAbs

| Clones | Binding epitopes | Manufacture | Reference |
|---|---|---|---|
| 5J7 | DII EDE | Creative biolab | [2] [3] |
| Mab 513 | DIII | Creative biolab | [4] |
| DV10 | DIII | Absolute antibody | [5] |
| DV18 | DI/DII | Absolute antibody | [5] |
| DV63 | DIII | Absolute antibody | [5] |
| DV78 | DI/DII | Absolute antibody | [5] |
| 4G2 | DII Fusion Loop | Absolute antibody | [6] |
| 777-3 (D6-8A1-12) | Envelope protein | In house prep | [7] |
| 78-2 | DII Fusion Loop | In house prep | [8] |

EDE: E dimer epitope
DI: Envelope protein Domain I,
DII: Domain II and
DIII: Domain III

### Animals and Immunizations

Two New Zealand White (NZW) female rabbits were immunized subcutaneously (S.Q.) with 200 μg DENV VLP with Freund's incomplete adjuvant or 200 μg DENV inactivated virus on day 0, 14, 28, and 42. The immunized rabbits collected the bleed on days 35 and 49 and confirmed the anti-sera titer against DENV VLP using Luminex assay or enzyme-linked immunosorbent assay (ELISA) neutralizing titer using RVP. One DENV2 immunized rabbit resumed immunization on day 120 and continued on day 140 with 200 μg DENV VLP with Freund's incomplete adjuvant. Selected rabbits were boosted with 400 μg DENV VLP or DENV inactivated virus by intravenous (IV) injection on day 59 or day 164. These immunized rabbits were sacrificed on day 64 or day 167 and collected spleen and bleeds. Spleen tissue was washed with an RPMI medium and dispersed to single cells by pipetting passed through a cell strainer. The single dispersed cell was conducted B cell sorting or stored in liquid nitrogen with a cell storage medium.

### Rabbit B Cell Sorting

For each sorting, ˜2×10⁸ freshly isolated splenocytes or 3 vials of thawed splenocytes (˜7×10⁷/vial) from selected rabbits were cultured in B-cell culture media (RPMI-1640, 15% FBS, 1×HEPES, 1×2-ME (1-Mercaptoethanol), 1% Penicillin/Streptomycin) overnight before sorting. 96-well B cell feeding plates were prepared one day before. Briefly, irradiated feeding cells in B cell culture media with a proprietary growth factor cocktail were dispensed into 96-well culture plates (120 μL/well). On the day of sorting, suspended and loosely attached splenocytes were collected by gently pipetting medium against the culturing surface of flask. The cells were then transferred to conical tube and spin at 400×g for 3 minutes. The cell pellets were washed with ice-cold FACS buffer (1×PBS+0.5% BSA) once. Then the biotinylated antigen was added at 5 μg/ml (final concentration). The mixture was incubated at R.T. for 20 min. The staining mixture was then centrifuged at 400×g for 3 min, and the cells were washed once in FACS buffer before being resuspended in FACS buffer and transferred into 1.5 ml amber Eppendorf tube. NeutrAvidin-Dy594 (1:300 dilution, Invitrogen cat #22842) and FITC-conjugated anti-rabbit IgM antibody (1:500 dilution, Novus, cat #MB7173) was then added, and the mixture was incubated at 4° C. for 15-30 min. The staining mixture was centrifuged at 400×g for 3 min. The cells were washed twice with ice-cold FACS buffer. The washed cell pellets were resuspended at ˜10⁷ cells/ml in ice-cold 1×PBS+1% FBS. At least 10 minutes before sorting, 7-AAD (1 μg/ml, final concentration) was added for live/dead cell determination. Single 7-AAD-/FITC-/Dy594+ cell was sorted into each well of a seeded 96-well plate. 96-well B cell culture plates with sorted B cells were cultured in 37° C. with 5% CO₂ for 9-12 days.

### Heavy-Chain and Light-Chain Variable Region (V.H. And V.L.) and Linier Expression Module (LEM) Construction and mAb Expression

Positive clones from B cell sorting supernatant screening were selected for V.H. and V.L. amplification by PCR, and LEM construction according to in-house SOP (Yurogen, Worcester, MA). Total RNAs from selected clones were purified from cell pellets preserved in DNA/RNA shield using RNeasy Mini Kit (Zymo, Cat #: R1051) following the manufacturer's protocol. Thirty-six μl nuclease-free water (Ambion, cat #AM9937) was used to elute total RNA. Eleven μl total RNA from each clone were mixed with 1 μl oligo (dT)12-18 primer (Invitrogen, Cat #58862) and 1 μl dNTPs (10 mM, ThermoScientific, cat #R0182) and then were heated at 65° C. for 5 min. Then for each clone mixture, 4 μl 5×First Strand buffer, 1 μl 100 mM DTT, 1 μl RNaseOUT (Invitrogen, Cat #: 10777-019), and 1 μl SuperScript III reverse transcriptase (Invitrogen, Cat #18080-044) was added. The reverse transcription reaction was carried out at 50° C. 1 hr and 75° C. 15 min to inactivate SuperScript III enzyme. After cDNA synthesis, V.H. and V.L. genes were amplified separately with V.H. and V.L. variable region primer pairs. The V.H. and VL PCR products were separated on 1% agarose gel electrophoresis system. The expected size of V.H. and V.L. amplicon is ˜500 bp. The corresponding bands of V.H. and V.L. for each clone were cut from gel, and V.H. and V.L. genes were extracted from gel with NucleoSpin® Gel and PCR Clean-Up Kit (Macherey-Nagel, Cat #740609.250) following manufacturer's protocol. Twelve—30 μl of elution buffer were used to elute V.H. and VL PCR products, depending on the amount of PCR products. The linear expression module cassette (LEM) PCR products were constructed by overlapping PCR with the C fragment containing CMV promotor, V.H. or V.L., and the H fragment contained rabbit IgG heavy-chain CH1-CH2-CH3 fragment, or light-chain constant region followed by SV40 transcription terminator and poly A signal sequences. Five 5 μl of PCR product were used to check the size and magnitude of amplification by 0.8% agarose gel electrophoresis. The remaining PCR products were purified with NucleoSpinR Gel and PCR Clean-Up Kit following manufacturer's protocol. Thirty-two μl of elution buffer was used to elute LEM PCR products. The LEM PCR products were transfected with 293E cells and collected supernatant post 4 day transfection for mAb screening.

### Cloning of V.H. And V.L. Into Expression Vector and Small Scale mAb Purification

V.H. and V.L. of selected clones were then cloned into the original expression vector pYURK using a one-step ligation independent cloning method. Plasmids with inserts were analyzed DNA sequences by Sanger sequencing methods. When multiple heavy or light chain constructs with different sequences were obtained for certain clones, transient expression of antibodies was performed using combinations of heavy and light chain constructs, and antigen-binding was confirmed for these supematants by ELISA. Full-length IgG heavy and light chain sequences (including signal peptides) were obtained from functional IgG heavy and light chain plasmids' sequences. IgG heavy and light chain DNA sequences with a signal peptide for secretion were cloned into pcDNA3.4 vector which was used for Expi293F cell transfection. Cell culture supernatant was collected, and antibodies in culture supernatant were affinity purified with Protein A agarose.

### Luminex Assay

The Luminex assay was conducted using FlexMap 3D (Luminex, Austin, TX, USA), and the conjugation of VLP was previously reported [9]. Briefly, 65 μg DENV proteins (Table 12) were conjugated to 1-Ethyl-3-[3-dimethylaminopropyl]carbodiimide hydrochloride, ECD/N-hydroxy-sulfo-succinimide, NHS (Thermo Fisher, Waltham, MA, USA) activated 12.5 million MagPlex beads (Luminex, Austin, TX, USA) at 50 mM 2-(N-morpholino) ethanesulfonic acid buffer pH 5.0-7.0 for 120 minutes at room temperature.

TABLE 12
VLP and Proteins for Luminex assay

| | | | Accession |
|---|---|---|---|
| Description | Source | Strain info | No |
| DENV-1 VLP | Native | Puerto Rico/US/BID- | ABO45246.1 |
| | Antigen | V853/1998 | |
| DENV-2 VLP | Native | Thailand/16681/84 | AAP06254.1 |
| | Antigen | | |
| DENV-3 VLP | Native | Sri Lanka D3/H/IMTSSA- | AXX75610.1 |
| | Antigen | SRI/2000/1266 | |
| DENV-4 VLP | Native | Dominica/814669/1981 | P09866.2 |
| | Antigen | | |
| ZIKV VLP | Native | Suriname Z1106033 | ALX35659.1 |
| | Antigen | | |
| Chikungunya | Native | Senegal 37997 | Q5XXP3.1 |
| virus VLP, | Antigen | | |
| Dengue Virus | Native | Thailand/16681/84 | AAB58782.1 |
| Serotype 2 | Antigen | | |
| envelope | | | |
| protein | | | |
| Dengue Virus | Native | Philippines/H241 | ALB78116.1 |
| Serotype 4 | Antigen | | |
| envelope | | | |
| protein | | | |
| Japanese | Native | SA-14 | P27395.1 |
| Encephalitis | Antigen | | |
| Virus VLP | | | |
| West Nile | Native | NY99 | ADD23575.1 |
| Virus | Antigen | | |
| envelope | | | |
| protein, | | | |

After conjugation, excess active residues were blocked by Sample buffer (1% bovine serum albumin (BSA) in Dulbecco's phosphate-buffered saline, D-PBS) overnight at 4° C. 10,000 DENV proteins conjugated beads/mL and anti-DENV mAb or anti-DENV mAb expressed supernatants were incubated at room temperature in sample buffer for 90 minutes and washed with phosphate-buffered saline plus 0.05% Tween-20 (PBST). After washing, the beads were incubated 10 μg/mL of Phycoerythrin-labeled anti-rabbit IgG (Thermo Fisher, Waltham, MA, USA) for 60 minutes. The beads were washed and mixed with Sheath Fluid (Luminex, Austin, TX, USA). The plates were measured the fluorescence intensity by FlexMap 3D.

k_{off} Ranking

The dissociation rate constant (k_{off}) of candidates' mAb bound to DENV-VLP was measured using Bio-layer interferometry (BLI) using an Octet-HTX (Sartorius, Fremont, CA, USA).

Briefly, antibody expressed supernatants were diluted with running buffer (0.1% Bovine Serum Albumin (BSA), PBS 0.05% Tween 20 (PBS-T)), and rabbit IgG was captured by Protein A biosensor (Sartorius, Fremont, CA, USA). The biosensors were transferred to 5 μg/mL of DENV-VLP solution for association (10 min) and then to a running buffer for dissociation. k_{off} of each mAbs was calculated by Octet Data Analysis Software H.T. (ver. 11.1.2.48 Sartorius, Fremont, CA, USA) with the Langmuir 1:1 binding model. The k_{off} for some serum samples could not be measured due to strong binding, in this case k_{off} were extrapolated to less than 2×10⁻⁵ (detectable dissociation from 0-1200 seconds for 5% signal decrease).

### Western Analysis

Western blot analysis was conducted by a capillary-based electrophoresis system [10](Wes, ProteinSimple, Santa Clara, CA, USA). In brief, DENV-VLP were denatured at 70° C. without reducing agent for 5 minutes, and VLP solution was loaded on a Wes assay plate and electrophoresed. Next, 10 μg/mL of Anti-DENV mAb were charged, followed by Wes horseradish peroxidase-conjugated anti-rabbit secondary antibody. The sample run was analyzed by examining the electropherogram and digital gel image.

### Reporter Virus-Particle (RVP) Assay

RVP assay was measured following the protocol from Bohning et al. [11]. Briefly, anti-DENV mAb expressed cell medium or serial diluted mAb solution were mixed with DENV RVP and the plate was incubated at 37° C. for 60 min in a 5% CO₂ humidified incubator. 4000-7500 cells/well Assay-Ready Raji DC-SIGN Cells (Accellerate, Hamburg, Germany) were added to the 384-well plates mAb/RVP mixture and incubated at 37° C. for 72 hrs in a 5% CO₂ incubator. Cell numbers were optimized following the manufacturer's instructions. To detect the Renilla luciferase activity in the cells, the plates were equilibrated to room temperature for 15 min and then incubated with Renilla-Glo™ Luciferase reagent according to the manufacturer's instructions (Promega, Madison, WI USA). Chemiluminescence were read with an EnSpire chemiluminescence reader (Perkin Elmer, Waltham MA USA).

### Construction of Anti-DENV mAb Expression Vectors

Each full length heavy-chain and light-chain DNA of anti-DENV mAb with a signal peptide were was synthesized and inserted to pcDNA3.4 mammalian expression vector. These antibody expression vectors were transformed to E. coli and amplified plasmid DNA was extracted, purified and sterilized for subsequent mammalian cell expression.

Antibody Expression and Purification of Anti-DENV mAbs

The light and heavy chain of rabbit mAb mammalian expression plasmids were co-transfected into Expi 293 cell systems (Thermo Fisher, Waltham, MA, USA) [12], and the transfected medium was harvested five days after transfection with centrifuging. Monoclonal antibodies were purified through rProtein A Sepharose (Cytiva, Marlborough, MA, USA). The eluted antibody was exchanged to Dulbecco's phosphate-buffered saline, D-PBS (Gibco, Waltham, MA, USA), using Amicon Ultra (Merck Millipore, Burlington, MA, USA). Antibody purity was determined by SDS-PAGE (NuPAGE, Thermo Fisher, Waltham, MA, USA) with heat-denatured, 70° C. for 10 min with reduced agents. Band intensity of SDS-PAGE was calculated by ChemoDoc Touch imaging system (BioRad, Hercules, CA USA).

### Antibody Expression Level Measurement

The antibody expression level was measured using Bio-layer interferometry (BLI) using an Octet-HTX (Sartorius, Fremont, CA, USA). Briefly, antibody expressed supematants and rabbit polyclonal antibody (Jackson ImmunoResearch Laboratories, West Grove, PA, USA) were diluted with running buffer (0.1% Bovine Serum Albumin (BSA), PBS 0.05% Tween 20 (PBS-T)), then these solutions were captured by Protein G biosensor (Sartorius, Fremont, CA, USA). The IgG concentration was calculated using rabbit IgG as a standard by Octet Data Analysis Software H.T. (ver. 11.1.2.48 Sartorius, Fremont, CA, USA).

### Allele Analysis

Anti-DENV mAb allele and CDR3 regions were analyzed by IMGT/V-QUEST (http://www.imgt.org/IMGT_vquest/analysis) or NCBI IGBLAST (https://www.ncbi.nlm.nih.gov/igblast/).

### Epitope Binning

Epitope binning was conducted by Octet-HTX (Forte Bio Fremont, CA, USA). Briefly, anti-DENV mAbs (20 μg/mL) were captured by ECD/Sulfo-NHS activated Amine Reactive Second Generation AR2Gbiosensor (Forte Bio Fremont, CA, USA). Next, 10 μg/mL anti-DENV mAbs were preincubated with 0.5 or 3 μg/mL DENV-VLP at room temperature for 10 min, and this mixture was bound to the antibody captured on the biosensor surface. These response data were subtracted by anti-DENV mAb only data. Binding activities were normalized to the response for DENV-VLP. Response signals of each pair were used for hierarchical clustering (Ward Method) using SAS JMP 13.1.0 (SAS, Cary, NC, USA).

### Data Analysis

EC₅₀ value of Luminex assay and IC₅₀ value for RVP assay were analyzed using GraphPad Prism (Ver.8.0.0, San Diego, CA USA).

### Results

Anti-DENV mAbs Screening from Rabbit

Anti-DENV3 serotype-specific mAbs were selected from 1920 well B cell sorting samples. Luminex assay and RVP assay were applied for B cell supernatant to select mAbs. For LEM products, high antigen reactivity and neutralization activity antibodies were selected by Luminex, RVP assay, and k_{off} ranking. Finally, anti-DENV antibodies with unique amino acid sequences have been chosen. The summary of the selection mAbs is shown in Table 13, and 11 mAb were selected for anti-DENV-3 type-specific mAbs.

TABLE 13
Summary of anti-DENV mAb screening

| | DENV3 |
|---|---|
| B cell sorting (N) | 1920 |
| B cell sorting positive clone (N) | 30 |
| LEM expression positive clone (N) | 19 |
| Final positive clones (N) | 11 |

### Antibody Sequence Analysis

Eleven anti-DENV-3 mAb alleles and CDR region sequences were analyzed by IMGT/V-QUEST. Heavy chain variable (V) region alleles were selected IGHVS40*01 and 45*01, and joining (J) region allele was only IGHJ4*01. Multiple diversity (D) region allele, IGHD1-1*01, 2-1*01, 6-1*01, 7-1*01 and 8-1*01 were selected (Table 14).

TABLE 14
Heavy chain allele gene analysis of anti-DENV3 mAbs

| Clone No | V Region | J Region | D Region |
|---|---|---|---|
| DENV3-5C6 | IGHV1S45*01 | IGHJ4*01 | IGHD6-1*01 |
| DENV3-5D7 | IGHV1S40*01 | IGHJ4*01 | IGHD8-1*01 |
| DENV3-8D4 | IGHV1S45*01 | IGHJ4*01 | IGHD6-1*01 |
| DENV3-10A3 | IGHV1S40*01 | IGHJ4*01 | IGHD1-1*01 |
| DENV3-12G7 | IGHV1S40*01 | IGHJ4*01 | IGHD8-1*01 |
| DENV3-12H6 | IGHV1S45*01 | IGHJ4*01 | IGHD2-1*01 |
| DENV3-13G3 | IGHV1S40*01 | IGHJ4*01 | IGHD1-1*01 |
| DENV3-13E7 | IGHV1S40*01 | IGHJ4*01 | IGHD1-1*01 |
| DENV3-13E10 | IGHV1S40*01 | IGHJ4*01 | IGHD8-1*01 |
| DENV3-18A4 | IGHV1S40*01 | IGHJ4*01 | IGHD1-1*01 |
| DEVN3-18C1 | IGHV1S45*01 | IGHJ4*01 | IGHD7-1*01 |

Light chain V region allele were dominant for IGKVT1S10*01 (5 clones/11 clones), IGKVT1S1*01, 3*02, 4*01, 12*01 and 22*01. All J region allele was IGKJ1-2*01 (Table 15).

TABLE 15
Light chain allele gene analysis of anti-DENV3 mAbs

| Clone No | V Region | J Region |
|---|---|---|
| DENV3-5C6 | IGKV1S67*01 | IGKJ1-2*01 |
| DENV3-5D7 | IGKV1S10*01 | IGKJ1-2*01 |
| DENV3_8D4 | IGKV1S52*01 | IGKJ1-2*01 |
| DENV3-10A3 | IGKV1S46*01 | IGKJ1-2*01 |
| DENV3-12G7 | IGKV1S4*01 | IGKJ1-2*01 |
| DENV3-12H6 | IGKV1S67*01 | IGKJ1-2*01 |
| DENV3-13G3 | IGKV1S37*01 | IGKJ1-2*01 |
| DENV3-13E7 | IGKV1S10*01 | IGKJ1-2*01 |
| DENV3-13E10 | IGKV1S36*01 | IGKJ1-2*01 |
| DENV3-18A4 | IGKV1S1*01 | IGKJ1-2*01 |
| DEVN3-18C1 | IGKV1S3*02 | IGKJ1-2*01 |

These antibodies showed unique CDR sequences.

Heavy chain CDR3 lengths were from 11 to 18 amino acid residues, and light chain CDR3

| | | measured conc. | | IgG conc |
|---|---|---|---|---|
| No | clone | μg/mL | dilution X | μg/mL |
| 1 | DENV3-5C6 | 21.67 | 10 | 217 |
| 2 | DENV3-5D7 | 25.10 | 10 | 251 |
| 3 | DENV3-8D4 | 43.44 | 10 | 434 |
| 4 | DENV3-10A3 | 26.59 | 10 | 266 |
| 5 | DENV3-12H6 | 23.40 | 10 | 234 |
| 6 | DENV3-12G7 | 16.59 | 10 | 166 |
| 7 | DENV3-13G3 | 59.34 | 10 | 593 |
| 8 | DENV3-13E7 | 0.67 | 1 | 0.67 |
| 9 | DENV3-13E10 | 28.44 | 10 | 284 |
| 10 | DENV3-18A4 | 28.16 | 10 | 282 |
| 11 | DENV3-18C1 | 17.63 | 10 | 176 |
| | no Ab | 0.096 | 1 | 0.096 |

lengths were 10-15 amino acid residues.

### Antibody Expression and Purification

Eleven anti-DENV3 mAb were expressed using Expi293 T expression system. Ten antibody clones showed high expression levels from 166 to 593 mg/L. However, Clone 13E7 showed a quite low antibody expression, 0.67 mg/L (Table 16).

### Table 16: Summary of Anti-DENV3 mAb Expression Levels

These ten anti-DENV3 mAbs were purified using rProtein A Sepharose. The purity of these mAbs was 100% confirmed by SDS-PAGE analysis.

### Antibody Binding to Dengue VLPs

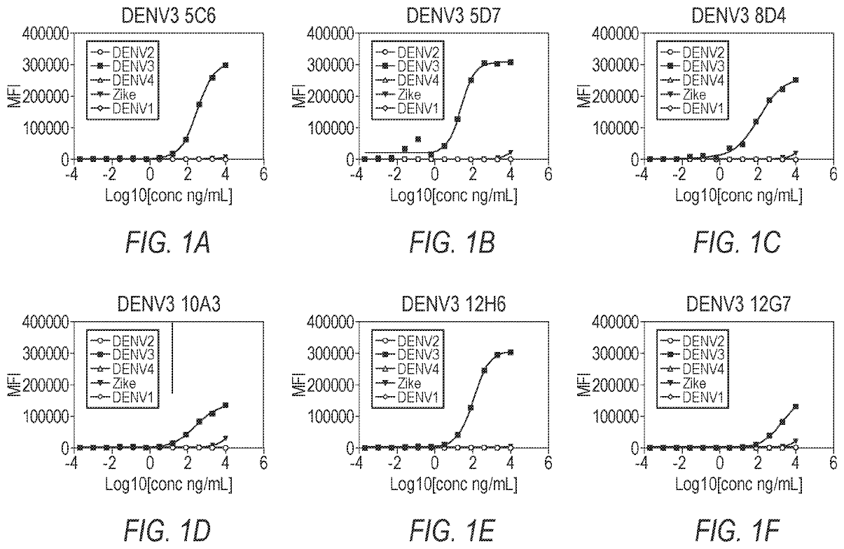

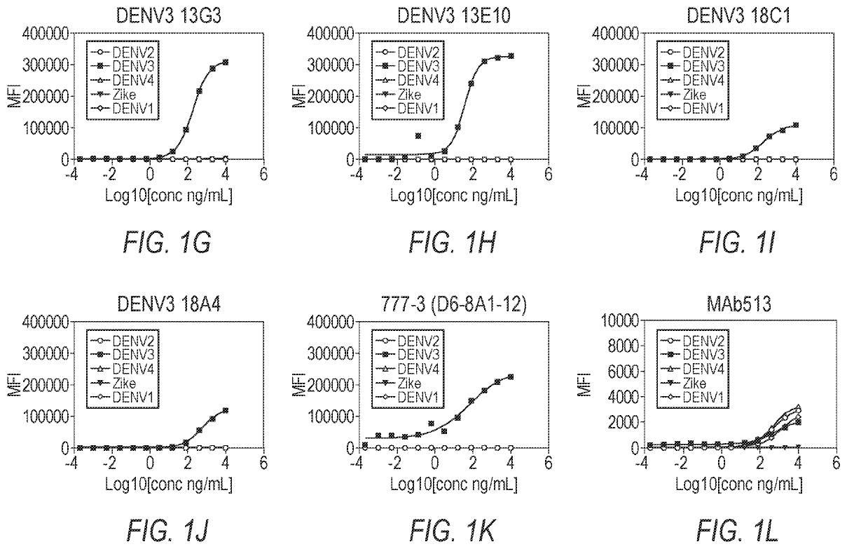

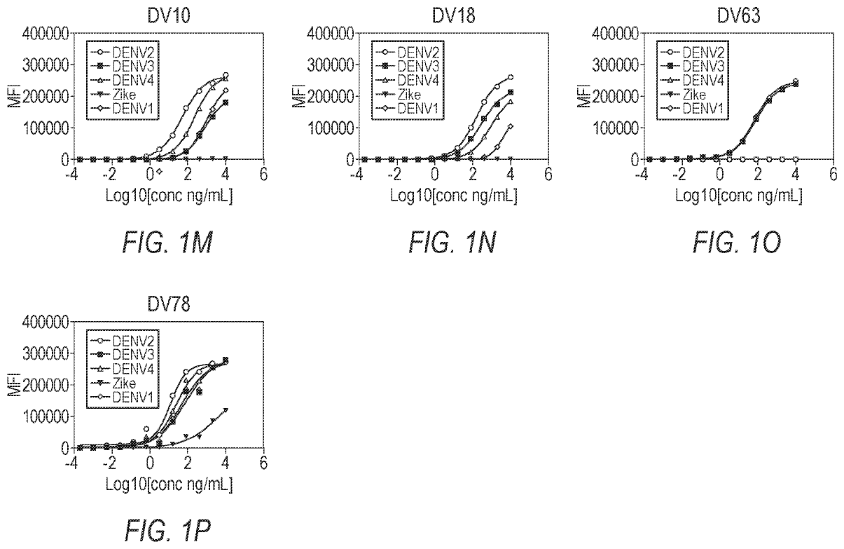

The binding activity of anti-DENV3 mAb was measured by Luminex. Ten mAb showed specific binding to dengue 3 VLP, and EC₅₀ values ranged from 25 to 1972 ng/mL. The EC₅₀ value of the clones 5D7 and 13E10 was smaller than the EC₅₀ value of the commercial clone mAbs. (FIGS. 1A-1P and Table 17)

TABLE 17
Summary of anti-DENV3 mAb reactivity

| | | | | Western |
|---|---|---|---|---|
| | | Luminex | | (band |
| DENV3- | | assay | RVP assay | at 0.6 ng |
| Clones | Specificity | EC₅₀ ng/mL | IC₅₀ nM | VLP/Lane) |
| DENV3-5C6 | DENV-3 | 320 | 2.781 | |
| DENV3-5D7 | DENV-3 | 25 | 12.209 | |
| DENV3-8D4 | DENV-3 | 115 | 0.760 | + |
| DENV3-10A3 | DENV-3 | 277 | 25.032 | +++ |
| DENV3-12H6 | DENV-3 | 110 | 0.339 | |
| DENV3-12G7 | DENV-3 | 1972 | N.D. | ++ |
| DENV3-13G3 | DENV-3 | 185 | 6.661 | ++ |
| DENV3-13E10 | DENV-3 | 36 | ND | |
| DENV3-18A4 | DENV-3 | 593 | 73.246 | |
| DENV3-18C1 | DENV-3 | 196 | ND | |
| 777-3 | DENV-3 | 68 | NT | |
| (D6-8A1-12) | | | | |
| Mab513 | CR | 694 | NT | |
| DV10 | CR | 846 | NT | |
| DV18 | CR | 282 | NT | |
| DV63 | DENV-1, 3 | 76 | NT | + |
| DV78 | CR | 77 | NT | |

ND: not detected,
NT: not tested,
CR: Cross Reactive

### Western Reactivity

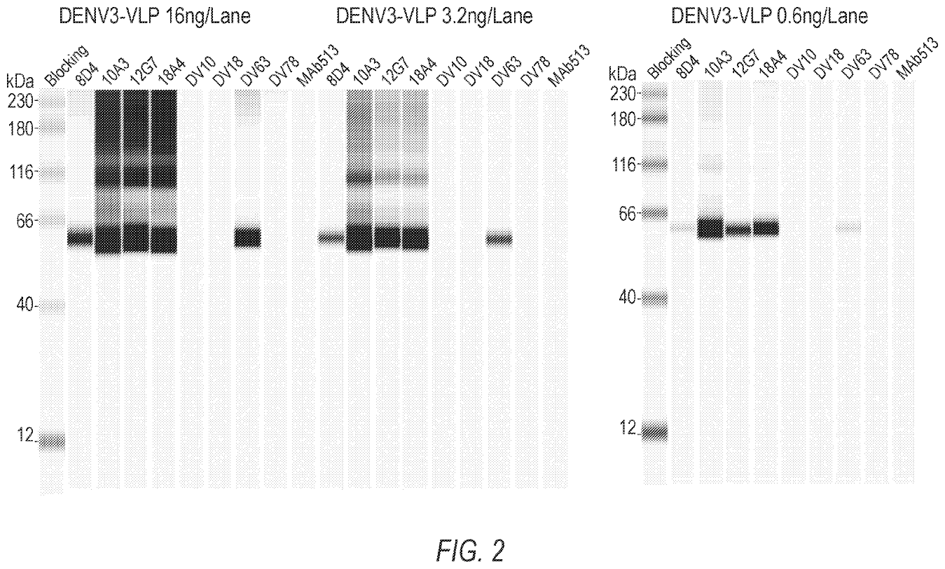

Four clones reacted to Western analysis. Clone 10A3 detected 0.6 ng DENV3 VLP by Western Blot analysis (FIG. 2 and Table 17). Commercial mAb, DV-63, also detected 3.2 ng DENV3 VLP. (FIG. 2).

Neutralizing Activity of Anti-DENV3 mAbs

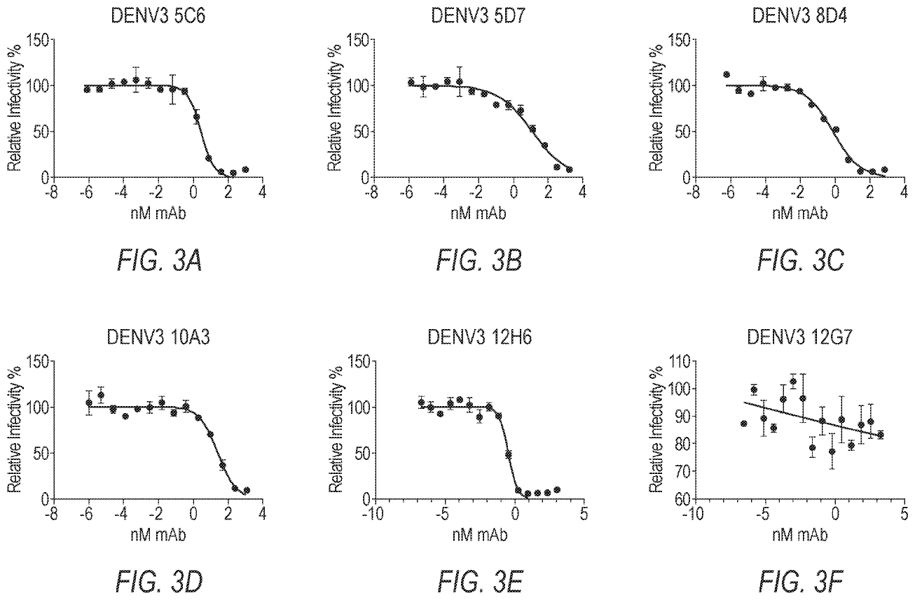

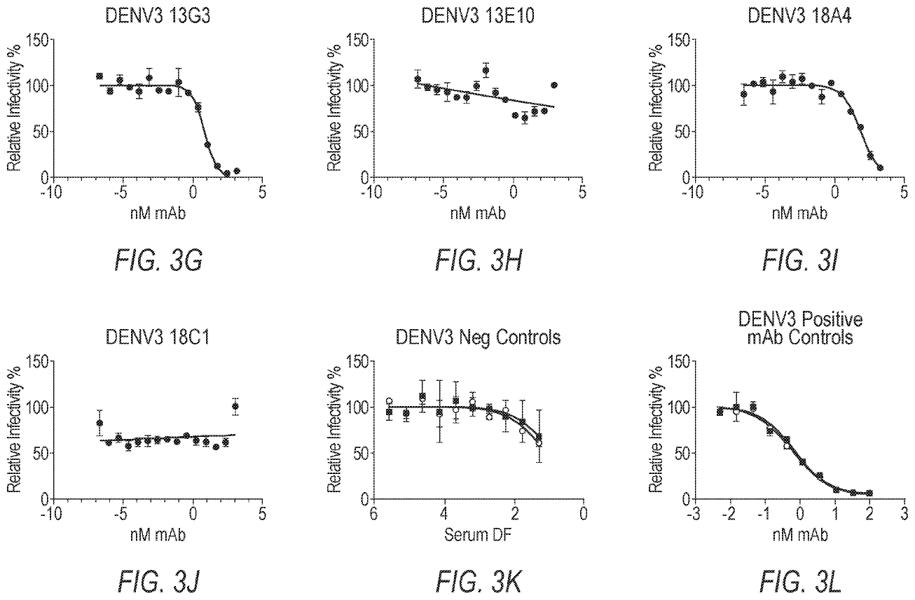

The neutralizing antibody activity was accessed by RVP assays. Two clones, DENV3-12H6 and 8D4 showed strong neutralizing activity. (FIGS. 3A-3L and Table 17).

Epitope Binning of Anti-DENV3 mAbs

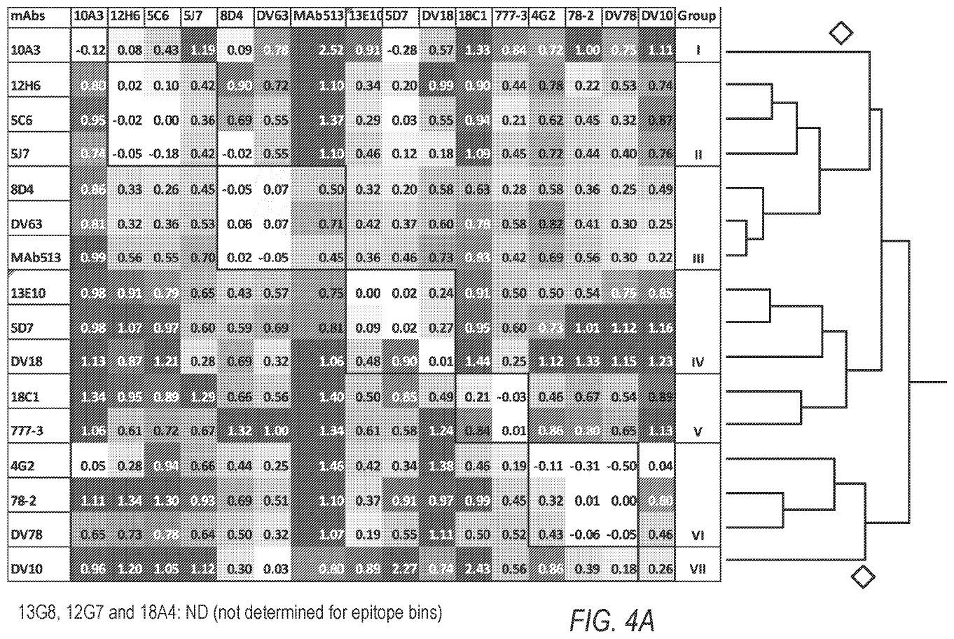

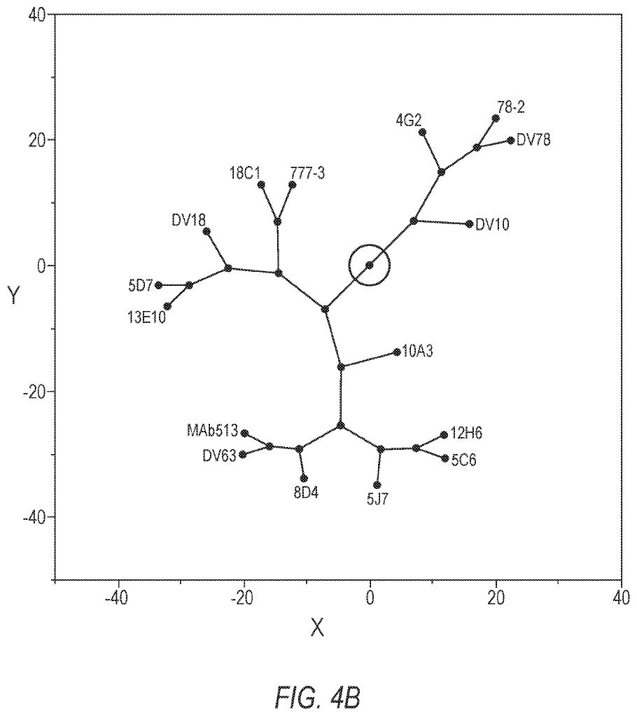

We conducted epitope binning analysis with ten in-house prepared mAbs and 7 commercial mAbs and two in-house prep mAbs. These mAbs were divided into 7 epitope bins. Clone 5C6 and 12H6 shared the same epitope bins of 5J7 bind to EDE epitopes. The EDE epitopes were unique and showed high neutralizing titers and 8D4 was same epitope bins with mAbs Domain III. The Domain III mAbs showed high neutralizing titers. Thus, these mAb can bind unique binding sites (FIGS. 4A and 4B).

k_{off} Ranking

The results of the k_{off} measurements for the different clones are shown in Table 18:

TABLE 18
k_{off} ranking

| | IgG conc | | |
|---|---|---|---|
| clones | ug/mL | response | k_{off} |
| DENV3-5C6 | 4.70 | 0.625 | <2.0E−05 |
| DENV3-5D7 | 5.16 | 0.984 | <2.0E−05 |
| DENV3-8D4 | 9.59 | 1.507 | <2.0E−05 |
| DENV3-10A3 | 11.66 | 0.339 | <2.0E−05 |
| DENV3-12H6 | 5.97 | 1.390 | <2.0E−05 |
| DENV3-12G7 | 7.11 | 0.158 | <2.0E−05 |
| DENV3-13G3 | 5.51 | 0.858 | <2.0E−05 |
| DENV3-13E7 | 2.88 | 1.659 | <2.0E−05 |
| DENV3-13D10 | 8.12 | 0.324 | <2.0E−05 |
| DENV3-18C1 | 3.95 | 0.383 | <2.0E−05 |
| DENV3-18A4 | 8.78 | 0.370 | <2.0E−05 |
| 4G2 | 5.00 | 0.316 | 3.45E−04 |

All of the antibodies according to the present invention exhibit a k_{off} rate which is less than the rate of the commercial antibody 4G2 [6].

### Detection of the Structure Changed Dengue Virus

### Methods

4G2 (1 μg/mL in 50 mM Bicarbonate buffer pH9.6; 100 μL/well) was coated with an ELISA plate (Maxisorp: Thermo Scientific) and stored in 4° C. overnight. Removed the 4G2 solution, washed the plate three times with PBS 0.05% Tween 20, then blocked the plate with 100 μL/well of SuperBlock T20 (PBS Blocking buffer (Thermo Scientific) at 37° C. for 60 min. Removed the SuperBlock T20 Blocking buffer, washed the plate three times with PBS 0.05% Tween 20, then 100 μL/well of various dilutions of Dengue 3 virus with pH4, 5, and 6 treated by HCl was added (started ×10 times dilutions with PBS 0.05% Tween 20) and incubated for 37° C. for 90 mm. Next, Removed the Dengue 3 virus, washed on the plate three times with PBS 0.05% Tween 20, then 100 μL/well of 1 μg/mL anti-DENV3 mAbs, DENV3-8D4, DENV3-10A3, and DENV3-12H6, in ×5 diluted SuperBlock T20 (PBS) Blocking buffer was added and incubated for 37° C. for 60 min. Removed the antibody solution and washed the plate three times with PBS 0.05% Tween 20, 100 μL/well of 1:5000 of diluted HRP-Labeled anti-rabbit IgG (H+L) (Jackson Immuno Research) in PBS 0.05% Tween 20 was added and incubated for 37° C. for 60 min. Removed the solution, washed the plate three times with PBS 0.05% Tween 20, then 100 μL/well of ABTS Peroxidase substrate (Sera Care) was added and incubated at room temperature for 15 min, then add 50 μL/well of KPL ABTS Peroxidase Stop Solution (Sera Care), then measure A405 nm of each well by Spectra Max (Molecular device).

### Results

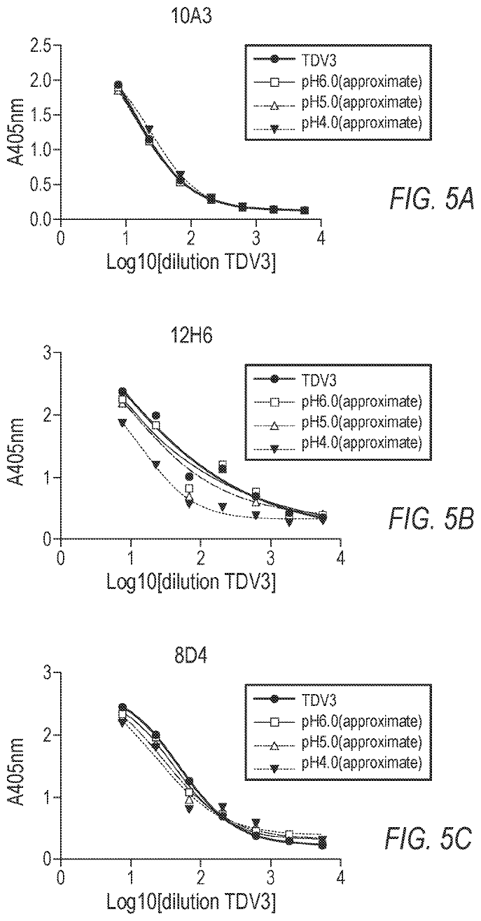

DENV3-10A3 did not change the binding between TDV (non-pH treated Dengue 3 virus) and pH-treated virus (FIG. 5). However, 12H6 showed weaker binding to pH 4 treated Dengue 3 virus. From the results, we conclude to monitor the structure change in Dengue 3 virus using 10A3 and 12H6 mAbs simultaneously.

Domain III Binding to Anti-Dengue 3 mAbs

### Methods

Anti-DENV3 mAb was diluted to 20 μg/mL 10 mM Acetate buffer pH5.0 or 4.0 (Sartorius), then 200 L/well of antibody solution were transferred to 96 well black plates (Griner) then coupled with AR2G biosensor (Sartorius) with EDC (1-Ethyl-3-[3-dimethylaminopropyl]carbodiimide hydrochloride) and S-NHS (N-hydroxysulfosuccinimide). The coupling biosensor was blocked by 200 μL/well of 1M Ethanol Amine solution pH8.5 (Sartorius), then adjusted the baseline with 200 μL/well sample buffer (0.1% BSA PBS 0.05% Tween20). 200 μL/well of 20 μg/mL DENV3 Domain III protein in sample buffer was incubated with an antibody-conjugated biosensor for 1200 sec. The response values at 1200 sec associated with Domain III protein were measured. The assay was done by Octet HTX (Sartorius) at 30° C.

### Results

DENV3-8D4 and DV63 and DV10 (Absolute antibody) observed ahigh Domain III binding response. Since DV63 and DV10 confirmed the domain III binding, thus we concluded DENV3-8D4 binds to Domain III (Table 19).

TABLE 19
Domain III binding to anti-Dengue 3 mAbs

| | | | TDV3 | |
|---|---|---|---|---|
| | | | Domain III | Response |
| Clones | domain | Reactivity | ug/mL | (nm) |
| DENV3-5C6 | NA | DENV3 | 20 | −0.0111 |
| DENV3-5D7 | NA | DENV3 | 20 | 0.0178 |
| DENV3-8D4 | Domain III | DENV3 | 20 | 0.2476 |
| DENV3-10A3 | NA | DENV3 | 20 | 0.0104 |
| DENV3-12H6 | NA | DENV3 | 20 | 0.0148 |
| DENV3-12G7 | NA | DENV3 | 20 | 0.0188 |
| DENV3-13G3 | NA | DENV3 | 20 | 0.0139 |
| DENV3-13E10 | NA | DENV3 | 20 | 0.0135 |
| DENV3-18A4 | NA | DENV3 | 20 | −0.0143 |
| DENV3-18C1 | NA | DENV3 | 20 | −0.0022 |
| DV63 | Domain III | DENV1, 3 | 20 | 0.2721 |
| DV10 | Domain III | CR | 20 | 0.2543 |
| DV78 | DI/DII | CR | 20 | −0.0082 |
| 78-2 | Fusion loop | CR | 20 | −0.0094 |
| DV3-E60 | Domain III | DENV3 | 20 | 0.2763 |
| MAb513 | DIII | CR | 20 | 0.2245 |
| 777-3 | NA | DENV3 | 20 | 0.1172 |

CR: cross-reactive to all 4 serotypes

### REFERENCES

1. Alen M M, Kaptein S J, De Burghgraeve T, Balzarini J, Neyts J, Schols D. Antiviral activity of carbohydrate-binding agents and the role of D C-SIGN in dengue virus infection. Virology 2009; 387:67-75.
2. Smith S A, Zhou Y, Olivarez N P, Broadwater A H, Silva A Md, Crowe J E. Persistence of Circulating Memory B Cell Clones with Potential for Dengue Virus Disease Enhancement for Decades following Infection. Journal of Virology 2012; 86:2665-75.
3. Fibriansah G, Tan J L, Smith S A, et al. A highly potent human antibody neutralizes dengue virus serotype 3 by binding across three surface proteins. Nature Communications 2015; 6:6341.
4. Wong Y H, Kumar A, Liew C W, et al. Molecular basis for dengue virus broad cross-neutralization by humanized monoclonal antibody 513. Sci Rep 2018; 8:8449.
5. Beltramello M, Williams K L, Simmons C P, et al. The human immune response to Dengue virus is dominated by highly cross-reactive antibodies endowed with neutralizing and enhancing activity. Cell Host Microbe 2010; 8:271-83.
6. Nawa M, Takasaki T, Yamada K I, Akatsuka T, Kurane I. Development of dengue IgM-capture enzyme-linked immunosorbent assay with higher sensitivity using monoclonal detection antibody. J Virol Methods 2001; 92:65-70.
7. Osorio J E, Brewoo J N, Silengo S J, et al. Efficacy of a tetravalent chimeric dengue vaccine (DENVax) in Cynomolgus macaques. Am J Trop Med Hyg 2011; 84:978-87.
8. Tsuji I, Vang F, Dominguez D, et al. Somatic Hypermutation and Framework Mutations of Variable Region Contribute to Anti-Zika Virus-Specific Monoclonal Antibody Binding and Function. Journal of Virology 2022; 96:e00071-22.
9. Nascimento E J M, Norwood B, Parker A, Braun R, Kpamegan E, Dean H J. Development and Characterization of a Multiplex Assay to Quantify Complement-Fixing Antibodies against Dengue Virus. Int J Mol Sci 2021; 22.
10. Harris V M. Protein detection by Simple Western™ analysis. Methods Mol Biol 2015; 1312:465-8.
11. Bohning K, Sonnberg S, Chen H L, et al. A high throughput reporter virus particle microneutralization assay for quantitation of Zika virus neutralizing antibodies in multiple species. PLoS One 2021; 16:e0250516.
12. Taki S, Kamada H, Inoue M, et al. A Novel Bispecific Antibody against Human CD3 and Ephrin Receptor A10 for Breast Cancer Therapy. PLoS One 2015; 10:e0144712.

### Items List

1. An antibody specific for Dengue virus serotype 3 (DENV3) or an antigen binding fragment thereof, wherein
(i) the VH CDR1 region of the antibody or antigen binding fragment thereof is selected from the group consisting of SEQ ID NO: 5, SEQ ID NO: 10, SEQ ID NO: 15 and SEQ ID NO: 20, or a variant thereof having at least 85% identity;
(ii) the VH CDR2 region of the antibody or binding fragment thereof is selected from the group consisting of SEQ ID NO: 6, SEQ ID NO: 11, SEQ ID NO: 16 and SEQ ID NO: 21, or a variant thereof having at least 85% identity;
(iii) the VH CDR3 region of the antibody or antigen binding fragment thereof is selected from the group consisting of SEQ ID NO: 7, SEQ ID NO: 12, SEQ ID NO: 17 and SEQ ID NO 22, or a variant thereof having at least 85% identity;
(iv) the VL CDR1 region of the antibody or antigen binding fragment thereof is selected from the group consisting of SEQ ID NO: 8, SEQ ID NO: 13, SEQ ID NO: 18 and SEQ ID NO: 23, or a variant thereof having at least 82% identity;
(v) the VL CDR2 region of the antibody or antigen binding fragment thereof is selected from the group of an amino acid sequence consisting of RAS, LAS and GAS, or a variant thereof having at least 65% identity; and
(vi) the VL CDR3 region of the antibody or antigen binding fragment thereof is selected from the group consisting of SEQ ID NO: 9, SEQ ID NO: 14, SEQ ID NO: 19 and SEQ ID NO: 24, or a variant thereof having at least 85% identity.

2. The antibody of item 1, wherein the amino acid sequence of the VH chain of the antibody or antigen binding fragment has an identity of at least 80% compared to the amino acid sequence set forth in SEQ ID NO: 3, preferably at least 90%; and even more preferred at least 95% sequence identity.

3. The antibody of item 1 or 2, wherein the amino acid sequence of the VL chain of the antibody or antigen binding fragment has an identity of at least 80% compared to the amino acid sequence set forth in SEQ ID NO: 4, preferably at least 90%; and even more preferred at least 95% sequence identity.

4. The antibody of any one of items 1 to 3, wherein the antibody of antigen binding fragment thereof does not cross-react with any dengue serotype other than DENV3, preferably, the antibody or antigen binding fragment does not cross-react with Zika virus.

5. The antibody of any one of items 1 to 4, wherein the antibody or antigen binding fragment thereof comprises the heavy chain CDR regions 1 to 3 having the amino acid sequences of SEQ ID Nos: 5 to 7, respectively, the light chain CDR region 1 has the amino acid sequence of SEQ ID NO: 8, the light chain CDR region 2 has the amino acid sequence RAS, and the light chain CDR region 3 has the amino acid sequence SEQ ID NO: 9.

6. The antibody of any one of items 1 to 5, wherein the antibody or antigen binding fragment thereof comprises the heavy chain CDR regions 1 to 3 having the amino acid sequences of SEQ ID Nos: 10 to 12, respectively, the light chain CDR region 1 has the amino acid sequence of SEQ ID NO: 13, the light chain CDR region 2 has the amino acid sequence LAS, and the light chain CDR region 3 has the amino acid sequence SEQ ID NO: 14.

7. The antibody of any one of items 1 to 6, wherein the antibody or antigen binding fragment thereof comprises the heavy chain CDR regions 1 to 3 having the amino acid sequences of SEQ ID Nos: 15 to 17, respectively, the light chain CDR region 1 has the amino acid sequence of SEQ ID NO: 18, the light chain CDR region 2 has the amino acid sequence RAS, and the light chain CDR region 3 has the amino acid sequence SEQ ID NO: 19

8. The antibody of any one of claims 1 to 7, wherein the antibody or antigen binding fragment thereof comprises the heavy chain CDR regions 1 to 3 having the amino acid sequences of SEQ ID Nos: 20 to 22, respectively, the light chain CDR region 1 has the amino acid sequence of SEQ ID NO: 23, the light chain CDR region 2 has the amino acid sequence GAS, and the light chain CDR region 3 has the amino acid sequence SEQ ID NO: 24.

9. The antibody of item 8, wherein the antibody or antigen binding fragment thereof comprises in the VH domain the amino acid sequence set forth in SEQ ID NO: 25 or a derivative thereof, and in the VL domain the amino acid sequence set forth in SEQ ID NO: 26 or a derivative thereof, wherein the derivative differs from the indicated sequence by not more than five amino acid mutations.

10. The antibody of item 9, wherein the antibody or antigen binding fragment thereof comprises in the VH domain the amino acid sequence set forth in SEQ ID NO: 27 or a derivative thereof, and in the VL domain the amino acid sequence set forth in SEQ ID NO: 28 or a derivative thereof, wherein the derivative differs from the indicated sequence by not more than five amino acid mutations.

11. The antibody of item 10, wherein the antibody or antigen binding fragment thereof comprises in the VH domain the amino acid sequence set forth in SEQ ID NO: 29 or a derivative thereof, and in the VL domain the amino acid sequence set forth in SEQ ID NO: 30 or a derivative thereof, wherein the derivative differs from the indicated sequence by not more than five amino acid mutations.

12. The antibody of item 11, wherein the antibody or antigen binding fragment thereof comprises in the VH domain the amino acid sequence set forth in SEQ ID NO: 31 or a derivative thereof, and in the VL domain the amino acid sequence set forth in SEQ ID NO: 32 or a derivative thereof, wherein the derivative differs from the indicated sequence by not more than five amino acid mutations.

1. An antibody specific for Dengue virus serotype 3 (DENV3) or an antigen binding fragment thereof, wherein
(i) the VH CDR1 region of the antibody or antigen binding fragment thereof is selected from the group consisting of SEQ ID NO: 5, SEQ ID NO: 10, SEQ ID NO: 15 and SEQ ID NO: 20, or a variant thereof having at least 85% identity;
(ii) the VH CDR2 region of the antibody or binding fragment thereof is selected from the group consisting of SEQ ID NO: 6, SEQ ID NO: 11, SEQ ID NO: 16 and SEQ ID NO: 21, or a variant thereof having at least 85% identity;
(iii) the VH CDR3 region of the antibody or antigen binding fragment thereof is selected from the group consisting of SEQ ID NO: 7, SEQ ID NO: 12, SEQ ID NO: 17 and SEQ ID NO 22, or a variant thereof having at least 85% identity;
(iv) the VL CDR1 region of the antibody or antigen binding fragment thereof is selected from the group consisting of SEQ ID NO: 8, SEQ ID NO: 13, SEQ ID NO: 18 and SEQ ID NO: 23, or a variant thereof having at least 82% identity;
(v) the VL CDR2 region of the antibody or antigen binding fragment thereof is selected from the group of an amino acid sequence consisting of RAS, LAS and GAS, or a variant thereof having at least 65% identity; and
(vi) the VL CDR3 region of the antibody or antigen binding fragment thereof is selected from the group consisting of SEQ ID NO: 9, SEQ ID NO: 14, SEQ ID NO: 19 and SEQ ID NO: 24, or a variant thereof having at least 85% identity,
wherein the antibody or antigen binding fragment thereof has one or more of the following properties:
(1) a neutralization activity calculated as IC₅₀ value of 15 nM or less;
(2) a binding activity for DENV3-VLP calculated as EC₅₀ value of 60 ng/ml or less; and/or
(3) a k_{off} value of 1×10⁻⁴ sec⁻¹ or less.

2. The antibody of claim 1, wherein the antibody or antigen binding fragment thereof has a neutralization activity calculated as IC₅₀ value of 5 nM or less, preferably, 2 nM or less, more preferably 1 nM or less.

3. The antibody of claim 1, wherein the antibody or antigen binding fragment thereof has a binding activity for DENV3-VLP calculated as EC₅₀ value of 50 ng/ml or less and more preferably 30 ng/ml or less.

4. The antibody of claim 1, wherein the antibody or antigen binding fragment thereof has a k_{off} value of 5×10⁻⁵ sec⁻¹ or less, preferably of 2×10⁻⁵ sec⁻¹ or less.

5. The antibody of claim 1, wherein the antibody or antigen binding fragment thereof has a neutralization activity calculated as IC₅₀ value of 0.5 nM or less and a k_{off} value of less than 5×10⁻⁵ sec⁻¹.

6. The antibody of claim 1, wherein the antibody or antigen binding fragment thereof has a binding activity for DENV3-VLP calculated as EC₅₀ value of 50 ng/ml or less and a k_{off} value of less than 5×10⁻⁵ sec⁻¹.

7. The antibody of claim 1, wherein the amino acid sequence of the VH chain of the antibody or antigen binding fragment has an identity of at least 80% compared to the amino acid sequence set forth in SEQ ID NO: 3, preferably at least 90%; and even more preferred at least 95% sequence identity.

8. The antibody of claim 1, wherein the amino acid sequence of the VL chain of the antibody or antigen binding fragment has an identity of at least 80% compared to the amino acid sequence set forth in SEQ ID NO: 4, preferably at least 90%; and even more preferred at least 95% sequence identity.

9. The antibody of claim 1, wherein the antibody or antigen binding fragment is specific for the dengue virus envelope protein, more preferably for domain III of the envelope protein.

10. The antibody of claim 1, wherein the antibody of antigen binding fragment thereof does not cross-react with any dengue serotype other than DENV3, preferably, the antibody or antigen binding fragment does not cross-react with Zika virus.

11. The antibody of claim 1, wherein the antibody or antigen binding fragment thereof comprises the heavy chain CDR regions 1 to 3 having the amino acid sequences of SEQ ID Nos: 5 to 7, respectively, the light chain CDR region 1 has the amino acid sequence of SEQ ID NO: 8, the light chain CDR region 2 has the amino acid sequence RAS, and the light chain CDR region 3 has the amino acid sequence SEQ ID NO: 9.

12. The antibody of claim 1, wherein the antibody or antigen binding fragment thereof comprises the heavy chain CDR regions 1 to 3 having the amino acid sequences of SEQ ID Nos: 10 to 12, respectively, the light chain CDR region 1 has the amino acid sequence of SEQ ID NO: 13, the light chain CDR region 2 has the amino acid sequence LAS, and the light chain CDR region 3 has the amino acid sequence SEQ ID NO: 14.

13. The antibody of claim 1, wherein the antibody or antigen binding fragment thereof comprises the heavy chain CDR regions 1 to 3 having the amino acid sequences of SEQ ID Nos: 15 to 17, respectively, the light chain CDR region 1 has the amino acid sequence of SEQ ID NO: 18, the light chain CDR region 2 has the amino acid sequence RAS, and the light chain CDR region 3 has the amino acid sequence SEQ ID NO: 19.

14. The antibody of claim 1, wherein the antibody or antigen binding fragment thereof comprises the heavy chain CDR regions 1 to 3 having the amino acid sequences of SEQ ID Nos: 20 to 22, respectively, the light chain CDR region 1 has the amino acid sequence of SEQ ID NO: 23, the light chain CDR region 2 has the amino acid sequence GAS, and the light chain CDR region 3 has the amino acid sequence SEQ ID NO: 24.

15. The antibody of claim 11, wherein the antibody or antigen binding fragment thereof comprises in the VH domain the amino acid sequence set forth in SEQ ID NO: 25 or a derivative thereof, and in the VL domain the amino acid sequence set forth in SEQ ID NO: 26 or a derivative thereof, wherein the derivative differs from the indicated sequence by not more than five amino acid mutations.

16. The antibody of claim 12, wherein the antibody or antigen binding fragment thereof comprises in the VH domain the amino acid sequence set forth in SEQ ID NO: 27 or a derivative thereof, and in the VL domain the amino acid sequence set forth in SEQ ID NO: 28 or a derivative thereof, wherein the derivative differs from the indicated sequence by not more than five amino acid mutations.

17. The antibody of claim 13, wherein the antibody or antigen binding fragment thereof comprises in the VH domain the amino acid sequence set forth in SEQ ID NO: 29 or a derivative thereof, and in the VL domain the amino acid sequence set forth in SEQ ID NO: 30 or a derivative thereof, wherein the derivative differs from the indicated sequence by not more than five amino acid mutations.

18. The antibody of claim 14-, wherein the antibody or antigen binding fragment thereof comprises in the VH domain the amino acid sequence set forth in SEQ ID NO: 31 or a derivative thereof, and in the VL domain the amino acid sequence set forth in SEQ ID NO: 32 or a derivative thereof, wherein the derivative differs from the indicated sequence by not more than five amino acid mutations.

19. The antibody of claim 1, wherein the neutralization activity is determined in a microneutralization (MNT) assay or reporter virus particle (RVP) assay, preferably the activity is determined in a RVP assay and the RVP is derived from DENV3 16562/Philippines/1964 (Accession No. U11673.1).

20. The antibody of claim 1, wherein the binding activity for DENV3-VLP is determined in an immunometric assay such as an ELISA, fluorescence or chemiluminescence assay, preferably DENV-3 VLP is derived from DENV3 strain Sri Lanka D3/H/IMTSSA-SRI/2000/1266 (Accession No. AXX75610.1), more preferably the assay is in Luminex assay format.

21. The antibody of claim 1, wherein the dissociation rate k_{off} value is determined by biolayer interferometry (BLI), preferably the dissociation rate of the antibody or antigen binding fragment thereof is determined with respect to DENV3-VLP.

22. The antibody of claim 1, wherein the antibody or antigen binding fragment thereof is selected from the group consisting of a Fab, F(ab)₂, Fv, single chain antibody, a chimeric antibody or a humanized antibody.

23. A pair of nucleic acids comprising a first nucleic acid encoding the heavy chain of the antibody of claim 1 and a second nucleic acid encoding the light chain of the antibody of claim 1.

24. A vector comprising the first nucleic acid of claim 23 under the control of one or more suitable promoters.

25. A host cell transformed with at least one vector of claim 24 and capable of expressing the antibody of claim 1.

26. A method for the recombinant production of the antibody of claim 1 comprising culturing the transformed host cell of claim 25 under conditions suitable of expressing the antibody of claim 1 and optionally purifying the antibody from the culture medium.

27. An in vitro method for detecting DENV3 viruses in a biological sample, wherein the method comprises contacting the antibody of claim 1 with a biological sample and determining the amount of antibody bound to the biological sample.

28. The in vitro method according to claim 27, wherein the antibody or antigen binding fragment thereof is not cross-reactive with Zika virus.

29. The in vitro method according to claim 27, wherein the in vitro method is a competitive assay.

30. A pharmaceutical formulation comprising the antibody of claim 1 and optionally one or more pharmaceutically acceptable carrier.

31. An antibody according to claim 1 for use in the prevention or treatment of a Dengue disease in a subject.

32. A method for the prevention or treatment of a Dengue disease in a subject comprising administering the antibody of claim 1 to the subject.

33. The antibody for use according to claim 38, wherein the subject is a human.

34. A kit for the detection of DENV3 viruses comprising an antibody of claim 1.


SEQUENCE LISTING (92 sequences)

SEQ ID NO: 1 - protein, 8 aa, Oryctolagus cuniculus
GFSFSSGY

SEQ ID NO: 2 - protein, 6 aa, Oryctolagus cuniculus
GXSGXT

SEQ ID NO: 3 - protein, 125 aa, unidentified
QXXLEESGGDLVKPGXSLTLTCXASGFSFSSXYXXCWVRQAPGKGLEWIXCIYXGXXXTXYAXWAKGRFTISKXSSTTVTLQXTSLTAADTATYFCARXXXXXXXXGXXXXFXLWGPGTLVTVSS

SEQ ID NO: 4 - protein, 111 aa, unidentified
AXVMTQTPSSVSXXVGGTVXINCQASESXXXXLAWYQQKPGQPPKLLIYXASXLXSGVPSRFXGSGSGTEFTLTISDXXCXDAATYYCXGXYXXSXXXXXXFGGGTEVVVK

SEQ ID NO: 5 - protein, 9 aa, Oryctolagus cuniculus
GFSFSNVYY

SEQ ID NO: 6 - protein, 8 aa, Oryctolagus cuniculus
IGTGDGNT

SEQ ID NO: 7 - protein, 18 aa, Oryctolagus cuniculus
ARDIYSYDNTGVYTVPKL

SEQ ID NO: 8 - protein, 8 aa, Oryctolagus cuniculus
ESVYSNNR

SEQ ID NO: 9 - protein, 11 aa, Oryctolagus cuniculus
AGGYSGSSDKG

SEQ ID NO: 10 - protein, 9 aa, Oryctolagus cuniculus
GIDFSSSYW

SEQ ID NO: 11 - protein, 8 aa, Oryctolagus cuniculus
IYTGSSTT

SEQ ID NO: 12 - protein, 12 aa, Oryctolagus cuniculus
ARFYDVGVYFNL

SEQ ID NO: 13 - protein, 6 aa, Oryctolagus cuniculus
ESISSW

SEQ ID NO: 14 - protein, 12 aa, Oryctolagus cuniculus
AGYKSSVTDGFA

SEQ ID NO: 15 - protein, 9 aa, Oryctolagus cuniculus
GFSFSSGYD

SEQ ID NO: 16 - protein, 9 aa, Oryctolagus cuniculus
IYITDTGSS

SEQ ID NO: 17 - protein, 14 aa, Oryctolagus cuniculus
AKTNYGSGGFAFNL

SEQ ID NO: 18 - protein, 6 aa, Oryctolagus cuniculus
QSISNL

SEQ ID NO: 19 - protein, 13 aa, Oryctolagus cuniculus
QCTYGGSSITNWA

SEQ ID NO: 20 - protein, 9 aa, Oryctolagus cuniculus
GFSFSSGSY

SEQ ID NO: 21 - protein, 8 aa, Oryctolagus cuniculus
IYSGGDVT

SEQ ID NO: 22 - protein, 20 aa, Oryctolagus cuniculus
ARGVGTYNYAGYAYPYYFSL

SEQ ID NO: 23 - protein, 6 aa, Oryctolagus cuniculus
ENVYGS

SEQ ID NO: 24 - protein, 12 aa, Oryctolagus cuniculus
QGAYYASNFDAT

SEQ ID NO: 25 - protein, 125 aa, Oryctolagus cuniculus
QEQLEESGGGLVKPGGTLTLTCTASGFSFSNVYYICWVRQAPGKGLEWIGCIGTGDGNTWYASWAKGRFTISKASSTTVILQVTSLTAADTATYFCARDIYSYDNTGVYTVPKLWGPGTLVTVSS

SEQ ID NO: 26 - protein, 111 aa, Oryctolagus cuniculus
AAVLTQTPSPVSAAVGGTVSISCQSSESVYSNNRLAWYQQKPGQPPKLLIYRASTLESGVPSRFKGSGSGTEFTLTISDVRCDDAATYYCAGGYSGSSDKGFGGGTEVVVK

SEQ ID NO: 27 - protein, 119 aa, Oryctolagus cuniculus
QEQLEESGGDLVKPEGSLTLTCKASGIDFSSSYWICWVRQAPGKGLEWIGCIYTGSSTTWYAAWAKGRFTISKPSSTTVTLQMTSLTAADTATYFCARFYDVGVYFNLWGPGTLVTVSS

SEQ ID NO: 28 - protein, 110 aa, Oryctolagus cuniculus
AIVMTQTPSSKSVPVGDTVTINCQASESISSWLAWYQQKPGQPPKLLIYLASTLASGVPSRFKGSGSGTQFTLTISDVVCDDAATYYCAGYKSSVTDGFAFGGGTEVVVK

SEQ ID NO: 29 - protein, 121 aa, Oryctolagus cuniculus
QSLEESGGDLVKPGASLTLTCTASGFSFSSGYDMCWVRQAPGKGLEWIACIYITDTGSSYYATWARGRFTISKTSSTSVTLQVTSLTAADTATYFCAKTNYGSGGFAFNLWGPGTLVTVSS

SEQ ID NO: 30 - protein, 111 aa, Oryctolagus cuniculus
DVVMTQTPASVEARVGGTVTINCQASQSISNLLAWYQQKPGQPPKLLIYRASALESGVPSRFRGSGSGTEFTLTISDLECADAATYYCQCTYGGSSITNWAFGGGTEVVVK

SEQ ID NO: 31 - protein, 126 aa, Oryctolagus cuniculus
QSLEESGGDLVKPGASLTLTCKASGFSFSSGSYMCWVRQAPGKGLDWIACIYSGGDVTYYTSWAKGRFTISRTSSTTVTLQMTSLTAADTATYFCARGVGTYNYAGYAYPYYFSLWGPGTLVTVSS

SEQ ID NO: 32 - protein, 110 aa, Oryctolagus cuniculus
ALVMTQTPSSVSEPVGGTVAINCQASENVYGSLAWYQQKPGQPPKLLIYGASYLASGVPSRFGGSGSGTEFTLTISDLECADAATYYCQGAYYASNFDATFGGGTEVVVK

SEQ ID NO: 33 - DNA, 27 bp, Oryctolagus cuniculus
  source 1..27: mol_type = genomic DNA
ggattctccttcagtaacgtctactac

SEQ ID NO: 34 - DNA, 24 bp, Oryctolagus cuniculus
  source 1..24: mol_type = genomic DNA
attggtactggtgatggcaacaca

SEQ ID NO: 35 - DNA, 54 bp, Oryctolagus cuniculus
  source 1..54: mol_type = genomic DNA
gcgagagatatatatagttatgataatactggtgtttatactgttcctaagttg

SEQ ID NO: 36 - DNA, 24 bp, Oryctolagus cuniculus
  source 1..24: mol_type = genomic DNA
gagagtgtttatagtaacaaccgc

SEQ ID NO: 37 - DNA, 33 bp, Oryctolagus cuniculus
  source 1..33: mol_type = genomic DNA
gcaggcggctatagtggtagtagtgataaaggt

SEQ ID NO: 38 - DNA, 27 bp, Oryctolagus cuniculus
  source 1..27: mol_type = genomic DNA
ggaatcgacttcagtagcagctactgg

SEQ ID NO: 39 - DNA, 24 bp, Oryctolagus cuniculus
  source 1..24: mol_type = genomic DNA
atttatactggtagtagtactaca

SEQ ID NO: 40 - DNA, 36 bp, Oryctolagus cuniculus
  source 1..36: mol_type = genomic DNA
gcgagattttatgatgtcggtgtttactttaacttg

SEQ ID NO: 41 - DNA, 18 bp, Oryctolagus cuniculus
  source 1..18: mol_type = genomic DNA
gagagcattagcagttgg

SEQ ID NO: 42 - DNA, 36 bp, Oryctolagus cuniculus
  source 1..36: mol_type = genomic DNA
gcaggatataaaagtagtgttactgatggttttgct

SEQ ID NO: 43 - DNA, 27 bp, Oryctolagus cuniculus
  source 1..27: mol_type = genomic DNA
ggattctccttcagtagcggctacgac

SEQ ID NO: 44 - DNA, 27 bp, Oryctolagus cuniculus
  source 1..27: mol_type = genomic DNA
atttatattactgatactggtagctct

SEQ ID NO: 45 - DNA, 42 bp, Oryctolagus cuniculus
  source 1..42: mol_type = genomic DNA
gcgaaaactaattatggtagtggtggttttgcctttaacttg

SEQ ID NO: 46 - DNA, 18 bp, Oryctolagus cuniculus
  source 1..18: mol_type = genomic DNA
cagagcattagcaacctc

SEQ ID NO: 47 - DNA, 39 bp, Oryctolagus cuniculus
  source 1..39: mol_type = genomic DNA
caatgcacttatggtggtagtagtattactaattgggct

SEQ ID NO: 48 - DNA, 27 bp, Oryctolagus cuniculus
  source 1..27: mol_type = genomic DNA
ggcttctccttcagtagcggctcatac

SEQ ID NO: 49 - DNA, 24 bp, Oryctolagus cuniculus
  source 1..24: mol_type = genomic DNA
atttatagtggtggtgatgtcact

SEQ ID NO: 50 - DNA, 60 bp, Oryctolagus cuniculus
  source 1..60: mol_type = genomic DNA
gcgagaggggtcggtacttataattatgctggttatgcttatccatactactttagcttg

SEQ ID NO: 51 - DNA, 18 bp, Oryctolagus cuniculus
  source 1..18: mol_type = genomic DNA
gagaacgtttacggctct

SEQ ID NO: 52 - DNA, 36 bp, Oryctolagus cuniculus
  source 1..36: mol_type = genomic DNA
caaggcgcttattatgccagtaattttgatgctact

SEQ ID NO: 53 - DNA, 376 bp, Oryctolagus cuniculus
  source 1..376: mol_type = genomic DNA
caggagcagctggaggagtccgggggaggcctggtcaagcctggaggaaccctgacactcacctgcacagcttctggattctccttcagtaacgtctactacatctgctgggtccgccaggctccagggaaggggctggaatggatcggatgcattggtactggtgatggcaacacatggtacgcgagctgggcgaaaggccgattcaccatctccaaagcctcgtcgaccacggtgattctgcaagtgaccagtctgacagccgcggacacggccacctatttctgtgcgagagatatatatagttatgataatactggtgtttatactgttcctaagttgtggggcccaggcaccctggtcaccgtctcctcag

SEQ ID NO: 54 - DNA, 334 bp, Oryctolagus cuniculus
  source 1..334: mol_type = genomic DNA
gccgccgtgctgacccagactccatctcccgtgtctgcagctgtgggaggcacagtcagcatcagttgccagtccagtgagagtgtttatagtaacaaccgcttagcctggtatcagcagaaaccagggcagcctcccaaactcctgatctacagggcatccactctggaatctggggtcccatcgcggttcaaaggcagtggatctgggacagagttcactctcaccatcagcgacgtgcggtgtgacgatgctgccacttattactgtgcaggcggctatagtggtagtagtgataaaggtttcggcggagggaccgaggtggtggtcaaag

SEQ ID NO: 55 - DNA, 358 bp, Oryctolagus cuniculus
  source 1..358: mol_type = genomic DNA
caggagcagctggaggagtccgggggagacctggtcaagcctgagggatccctgacactcacctgcaaagcctctggaatcgacttcagtagcagctactggatatgctgggtccgccaggctccagggaaggggctggagtggatcggatgcatttatactggtagtagtactacatggtacgcggcctgggcgaaaggccgattcaccatctccaagccctcgtcgaccacggtgactctgcaaatgaccagtctgacggccgcggacacggccacgtatttctgtgcgagattttatgatgtcggtgtttactttaacttgtggggcccaggcaccctggtcaccgtctcctcag

SEQ ID NO: 56 - DNA, 331 bp, Oryctolagus cuniculus
  source 1..331: mol_type = genomic DNA
gccatcgtgatgacccagactccatcttccaagtctgtccctgtgggagacacagtcaccatcaattgccaggccagtgagagcattagcagttggttagcctggtatcagcagaaaccagggcagcctcccaagctcctgatctatctggcatccactctggcatctggggtcccatcgcggttcaaaggcagtggatctgggacacagttcactctcaccatcagcgatgtggtgtgtgacgatgctgccacttactactgtgcaggatataaaagtagtgttactgatggttttgctttcggcggagggaccgaggtggtggtcaaag

SEQ ID NO: 57 - DNA, 364 bp, Oryctolagus cuniculus
  source 1..364: mol_type = genomic DNA
cagtcgttggaggagtccgggggagacctggtcaagcctggggcatccctgacactcacctgcacagcctctggattctccttcagtagcggctacgacatgtgctgggtccgccaggctccagggaaggggttggagtggatcgcatgcatttatattactgatactggtagctcttattacgcgacctgggcgagaggccgattcaccatctccaaaacctcgtcgacctcggtgactctgcaagtgaccagtctgacagccgcggacacggccacctatttctgtgcgaaaactaattatggtagtggtggttttgcctttaacttgtggggcccaggcaccctggtcaccgtctcctcag

SEQ ID NO: 58 - DNA, 334 bp, Oryctolagus cuniculus
  source 1..334: mol_type = genomic DNA
gatgttgtgatgacccagactccagcctccgtggaggcacgtgtgggaggcacagtcaccatcaattgccaggccagtcagagcattagcaacctcttagcctggtatcagcagaaaccagggcagcctcccaagctcctgatctacagggcttccgctctggaatctggggtcccgtcgcggttcagaggcagtggatctgggacagagttcactctcaccatcagcgacctggagtgtgccgatgctgccacttactactgtcaatgcacttatggtggtagtagtattactaattgggctttcggcggagggaccgaggtggtggtcaaag

SEQ ID NO: 59 - DNA, 379 bp, Oryctolagus cuniculus
  source 1..379: mol_type = genomic DNA
cagtcgttggaggagtccgggggagacctggtcaagcctggggcatccctgacactcacctgcaaagcctctggcttctccttcagtagcggctcatacatgtgctgggtccgccaggctccagggaaggggctggattggatcgcatgcatttatagtggtggtgatgtcacttactacacgagctgggcgaaaggccgattcaccatctccagaacctcgtcgaccacggtgactctgcaaatgaccagtctgacagccgcggacacggccacctatttctgtgcgagaggggtcggtacttataattatgctggttatgcttatccatactactttagcttgtggggcccaggcaccctggtcaccgtctcctcag

SEQ ID NO: 60 - DNA, 331 bp, Oryctolagus cuniculus
  source 1..331: mol_type = genomic DNA
gcccttgtgatgacccagactccatcctccgtgtctgaacctgtgggaggcacagtcgccatcaattgccaggccagtgagaacgtttacggctctttagcctggtatcagcagaaaccagggcagcctcccaagctcctgatctatggtgcatcctatctggcatctggggtcccatcgcggttcggtggcagtggatctgggacagagttcactctcaccatcagcgacctggagtgtgccgatgcagccacttactactgtcaaggcgcttattatgccagtaattttgatgctactttcggcggagggaccgaggtggtggtcaaag

SEQ ID NO: 61 - protein, 24 aa, Oryctolagus cuniculus
QSLEESGGDLVKPGASLTLTCTAS

SEQ ID NO: 62 - protein, 17 aa, Oryctolagus cuniculus
MCWVRQAPGKGLEWIAC

SEQ ID NO: 63 - protein, 37 aa, Oryctolagus cuniculus
YYATWARGRFTISKTSSTSVTLQVTSLTAADTATYFC

SEQ ID NO: 64 - protein, 11 aa, Oryctolagus cuniculus
WGPGTLVTVSS

SEQ ID NO: 65 - protein, 26 aa, Oryctolagus cuniculus
DVVMTQTPASVEARVGGTVTINCQAS

SEQ ID NO: 66 - protein, 17 aa, Oryctolagus cuniculus
LAWYQQKPGQPPKLLIY

SEQ ID NO: 67 - protein, 36 aa, Oryctolagus cuniculus
ALESGVPSRFRGSGSGTEFTLTISDLECADAATYYC

SEQ ID NO: 68 - protein, 10 aa, Oryctolagus cuniculus
FGGGTEVVVK

SEQ ID NO: 69 - protein, 25 aa, Oryctolagus cuniculus
QEQLEESGGGLVKPGGTLTLTCTAS

SEQ ID NO: 70 - protein, 17 aa, Oryctolagus cuniculus
ICWVRQAPGKGLEWIGC

SEQ ID NO: 71 - protein, 37 aa, Oryctolagus cuniculus
WYASWAKGRFTISKASSTTVILQVTSLTAADTATYFC

SEQ ID NO: 72 - protein, 11 aa, Oryctolagus cuniculus
WGPGTLVTVSS

SEQ ID NO: 73 - protein, 26 aa, Oryctolagus cuniculus
AAVLTQTPSPVSAAVGGTVSISCQSS

SEQ ID NO: 74 - protein, 17 aa, Oryctolagus cuniculus
LAWYQQKPGQPPKLLIY

SEQ ID NO: 75 - protein, 36 aa, Oryctolagus cuniculus
TLESGVPSRFKGSGSGTEFTLTISDVRCDDAATYYC

SEQ ID NO: 76 - protein, 10 aa, Oryctolagus cuniculus
FGGGTEVVVK

SEQ ID NO: 77 - protein, 25 aa, Oryctolagus cuniculus
QEQLEESGGDLVKPEGSLTLTCKAS

SEQ ID NO: 78 - protein, 17 aa, Oryctolagus cuniculus
ICWVRQAPGKGLEWIGC

SEQ ID NO: 79 - protein, 37 aa, Oryctolagus cuniculus
WYAAWAKGRFTISKPSSTTVTLQMTSLTAADTATYFC

SEQ ID NO: 80 - protein, 11 aa, Oryctolagus cuniculus
WGPGTLVTVSS

SEQ ID NO: 81 - protein, 26 aa, Oryctolagus cuniculus
AIVMTQTPSSKSVPVGDTVTINCQAS

SEQ ID NO: 82 - protein, 17 aa, Oryctolagus cuniculus
LAWYQQKPGQPPKLLIY

SEQ ID NO: 83 - protein, 36 aa, Oryctolagus cuniculus
TLASGVPSRFKGSGSGTQFTLTISDVVCDDAATYYC

SEQ ID NO: 84 - protein, 10 aa, Oryctolagus cuniculus
FGGGTEVVVK

SEQ ID NO: 85 - protein, 24 aa, Oryctolagus cuniculus
QSLEESGGDLVKPGASLTLTCKAS

SEQ ID NO: 86 - protein, 17 aa, Oryctolagus cuniculus
MCWVRQAPGKGLDWIAC

SEQ ID NO: 87 - protein, 37 aa, Oryctolagus cuniculus
YYTSWAKGRFTISRTSSTTVTLQMTSLTAADTATYFC

SEQ ID NO: 88 - protein, 11 aa, Oryctolagus cuniculus
WGPGTLVTVSS

SEQ ID NO: 89 - protein, 26 aa, Oryctolagus cuniculus
ALVMTQTPSSVSEPVGGTVAINCQAS

SEQ ID NO: 90 - protein, 17 aa, Oryctolagus cuniculus
LAWYQQKPGQPPKLLIY

SEQ ID NO: 91 - protein, 36 aa, Oryctolagus cuniculus
YLASGVPSRFGGSGSGTEFTLTISDLECADAATYYC

SEQ ID NO: 92 - protein, 10 aa, Oryctolagus cuniculus
FGGGTEVVVK